SEQUENCE LISTING (7 sequences)

SEQ ID NO: 1 - protein, 165 aa, Homo sapiens
WSNNYAVDCPQHCDSSECKSSPRCKRTVLDDCGCCRVCAAGRGETCYRTVSGMDGMKCGPGLRCQPSNGEDPFGEEFGICKDCPYGTFGMDCRETCNCQSGICDRGTGKCLKFPFFQYSVTKSSNRFVSLTEHDMASGDGNIVREEVVKENAAGSPVMRKWLNPR

SEQ ID NO: 2 - DNA, 2006 bp, Homo sapiens
cttcccaccagcaaagaccacgactggagagccgagccggaggcagctgggaaacatgaagagcgtcttgctgctgaccacgctcctcgtgcctgcacacctggtggccgcctggagcaataattatgcggtggactgccctcaacactgtgacagcagtgagtgcaaaagcagcccgcgctgcaagaggacagtgctcgacgactgtggctgctgccgagtgtgcgctgcagggcggggagaaacttgctaccgcacagtctcaggcatggatggcatgaagtgtggcccggggctgaggtgtcagccttctaatggggaggatccttttggtgaagagtttggtatctgcaaagactgtccctacggcaccttcgggatggattgcagagagacctgcaactgccagtcaggcatctgtgacagggggacgggaaaatgcctgaaattccccttcttccaatattcagtaaccaagtcttccaacagatttgtttctctcacggagcatgacatggcatctggagatggcaatattgtgagagaagaagttgtgaaagagaatgctgccgggtctcccgtaatgaggaaatggttaaatccacgctgatcccggctgtgatttctgagagaaggctctattttcgtgattgttcaacacacagccaacattttaggaactttctagatatagcataagtacatgtaatttttgaagatccaaattgtgatgcatggtggatccagaaaacaaaaagtaggatacttacaatccataacatccatatgactgaacacttgtatgtgtttgttaaatattcgaatgcatgtagatttgttaaatgtgtgtgtatagtaacactgaagaactaaaaatgcaatttaggtaatcttacatggagacaggtcaaccaaagagggagctaggcaaagctgaagaccgcagtgagtcaaattagttctttgactttgatgtacattaatgttgggatatggaatgaagacttaagagcaggagaagatggggagggggtgggagtgggaaataaaatatttagcccttccttggtaggtagcttctctagaatttaattgtgctttttttttttttttggctttgggaaaagtcaaaataaaacaaccagaaaacccctgaaggaagtaagatgtttgaagcttatggaaatttgagtaacaaacagctttgaactgagagcaatttcaaaaggctgctgatgtagttcccgggttacctgtatctgaaggacggttctggggcataggaaacacatacacttccataaatagctttaacgtatgccacctcagagataaatctaagaagtattttacccactggtggtttgtgtgtgtatgaaggtaaatatttatatatttttataaataaatgtgttagtgcaagtcatcttccctacccatatttatcatcctcttgaggaaagaaatctagtattatttgttgaaaatggttagaataaaaacctatgactctataaggttttcaaacatctgaggcatgataaatttattatccataattataggagtcactctggatttcaaaaaatgtcaaaaaatgagcaacagagggaccttatttaaacataagtgctgtgacttcggtgaattttcaatttaaggtatgaaaataagtttttaggaggtttgtaaaagaagaatcaattttcagcagaaaacatgtcaactttaaaatataggtggaattaggagtatatttgaaagaatcttagcacaaacaggactgttgtactagatgttcttaggaaatatctcagaagtattttatttgaagtgaagaacttatttaagaattatttcagtatttacctgtattttattcttgaagttggccaacagagttgtgaatgtgtgtggaaggcctttgaatgtaaagctgcataagctgttaggttttgttttaaaaggacatgtttattattgttcaataaaaaagaacaagatac

SEQ ID NO: 3 - DNA, 555 bp, Artificial Sequence
  Obtained by directed-mutagenesis
atgaagagcgtcttgctgctgaccacgctcctcgtgcctgcacacctggtggccgcctggagcaataattatgcggtggactgccctcaacactgtgacagcagtgagtgcaaaagcagcccgcgctgcgagaggacagtgctcgacgactgtggctgctgccgagtgtgcgctgcagggcggggagaaacttgctaccgcacagtctcaggcatggatggcatgaagtgtggcccggggctgaggtgtcagccttctaatggggaggatccttttggtgaagagtttggtatctgcaaagactgtccctacggcaccttcgggatggattgcagagagacctgcaactgccagtcaggcatctgtgacagggggacgggaaaatgcctgtaattccccttcgcccaatattcagtaaccaagtcttccaacagatttgtttctctcacggagcatgacatggcatctggagatggcaatattgtgagagaagaagttgtgaaagagaatgctgccgggtctcccgtaatgaggaaatggttaaatccacgctga

SEQ ID NO: 4 - DNA, 555 bp, Artificial Sequence
  Obtained by directed-mutagenesis
atgaagagcgtcttgctgctgaccacgctcctcgtgcctgcacacctggtggccgcctggagcaataattatgcggtggactgccctcaacactgtgacagcagtgagtgcaaaagcagcccgcgctgcgagaggacagtgctcgacgactgtggctgctgccgagtgtgcgctgcagggcggggagaaacttgctaccgcacagtctcaggcatggatggcatgaagtgtggcccggggctgaggtgtcagccttctaatggggaggatccttttggtgaagagtttggtatctgcaaagactgtccctacggcaccttcgggatggattgcagagagacctgcaactgccagtcaggcatctgtgacagggggacgggaaaatgcctgaaattccccttctaacaatattcagtaaccaagtcttccaacagatttgtttctctcacggagcatgacatggcatctggagatggcaatattgtgagagaagaagttgtgaaagagaatgctgccgggtctcccgtaatgaggaaatggttaaatccacgctga

SEQ ID NO: 5 - DNA, 555 bp, Artificial Sequence
  Obtained by directed-mutagenesis
atgaagagcgtcttgctgctgaccacgctcctcgtgcctgcacacctggtggccgcctggagcaataattatgcggtggactgccctcaacactgtgacagcagtgagtgcaaaagcagcccgcgctgcgagaggacagtgctcgacgactgtggctgctgccgagtgtgcgctgcagggcggggagaaacttgctaccgcacagtctcaggcatggatggcatgaagtgtggcccggggctgaggtgtcagccttctaatggggaggatccttttggtgaagagtttggtatctgcaaagactgtccctacggcaccttcgggatggattgcagagagacctgcaactgccagtcaggcatctgtgacagggggacgggaaaatgcctgaaattccccttcttctaatattcagtaaccaagtcttccaacagatttgtttctctcacggagcatgacatggcatctggagatggcaatattgtgagagaagaagttgtgaaagagaatgctgccgggtctcccgtaatgaggaaatggttaaatccacgctga

SEQ ID NO: 6 - protein, 13 aa, Artificial Sequence
  Synthetic peptide
DRGTGKCLKFPFF

SEQ ID NO: 7 - protein, 9 aa, Artificial Sequence
  Synthetic peptide
GKCLKFPFF

(19) United States
(12) Patent Application Publication
(10) Pub. No.: US 2012/0015387 A1
(43) Pub. Date: Jan. 19, 2012

(54) MARKER PEPTIDES FOR DETERMINING THE OCCURRENCE OF AN INFLAMMATORY STATE IN A SUBJECT

(75) Inventors: Nathalie De Freitas Caires, Lille Cedex (FR); Philippe Lassalle, Lille Cedex (FR); Philippe Hauw, Lille Cedex (FR)

(73) Assignees: INSERM (INSTITUT NATIONAL DE LA SANTE ET DE LA RECHERCHE MEDICALE), Paris (FR); INSTITUT PASTEUR DE LILLE, Lille (FR)

(21) Appl. No.: 13/123,028

(22) PCT Filed: Oct. 8, 2009
(86) PCT No.: PCT/EP2009/007246

§ 371 Date: Apr. 7, 2011

(30) Foreign Application Priority Data
Oct. 8, 2008 (EP) 08305655.6

(51) Int. Cl. G01N 33/577; H01J 49/26; C07K 16/18; G01N 33/566; C07K 14/435

(52) U.S. Cl. 435/7.94; 436/501; 530/350; 530/388.1; 250/282

(57) ABSTRACT

An enzyme proteolysis-resistant peptide that binds to antibodies directed against the amino acid region 1-116 of the endocan's polypeptide sequence, which peptide possesses an apparent molecular weight of 14 kDa.

### FIELD OF THE INVENTION

The present invention concerns the field of proteomics, and in particular diagnosis of the occurrence of an inflammatory state in a subject.

### BACKGROUND OF THE INVENTION

Inflammation is the complex biological response of vascular tissues to harmful stimuli, such as pathogens, damaged cells, or irritants. It is a protective attempt by the organism to remove the injurious stimuli as well as initiate the healing process for the tissue.

Inflammation can be classified as either acute or chronic.

Acute inflammation is the initial response of the body to harmful stimuli and is achieved by the increased movement of plasma and leukocytes from the blood into the injured tissues. A cascade of biochemical events propagates and matures the inflammatory response, involving the local vascular system, the immune system and various cells within the injured tissue.

Prolonged inflammation, known as chronic inflammation, leads to a progressive shift in the type of cells which are present at the site of inflammation and is characterised by simultaneous destruction and healing of the tissue from the inflammatory process.

For example, sepsis is a serious medical condition characterized by a whole-body inflammatory state caused by infection. According to the standard defined by the American College of Chest Physicians/Society of Critical Care Medicine Consensus Conference (1992, definitions for sepsis and multiple organ failure, and guidelines for the use of innovative therapies in sepsis; Crit Care Med., vol. 20: 864-874), sepsis may be divided into three categories of increasing severity: sepsis, severe sepsis and septic shock.

Sepsis may provoke Acute Lung Injury (ALI) and Acute Respiratory Distress Syndrome (ARDS), which represent major cause of respiratory failure and require intensive care and life support.

One key event in the early development of ALI/ARDS is the alteration of the alveolar-capillary membrane, mainly resulting from polymorphonuclear neutrophils leukocytes (PMN) sequestration in the pulmonary capillaries, PMN emigration and endothelial cell injury.

ARDS occurrence is about of 15 to 34 cases per 100 000 inhabitants in the USA. The 28-days mortality is estimated between 20 to 40% according to the studies.

In France, 250 000 people are admitted, each year, in intensive care units. Among them, 54 000 are in sepsis choc state, the severest form.

The development of sepsis in a patient is usually monitored by the quantification of three markers, respectively C-reactive protein, soluble ICAM-1 protein and procalcitonin.

Another potentially really interesting biomarker of inflammatory state is protein Endocan.

Endocan is a proteoglycan consisting of a protein core of 20 kDa and a unique GAG chain of chondroitin sulphate/dermatan sulphate, O-linked to the serine 137. Endocan is spontaneously and preferentially expressed by lung endothelial cells. This glycosylated protein circulates with a mean amount level around 1 ng/mL in the bloodstream. Its synthesis and secretion by human umbilical vein endothelial cells (HUVECs) are up-regulated by proinflammatory cytokines TNFα and IL-1.

Protein endocan, due to its role in the regulation of inflammatory reactions, constitutes potentially a marker of the development of sepsis whose physiological significance is directly linked to the uncontrolled development of the inflammatory reaction, particularly the recruitment of leukocytes during the phenomena of extravasation and massive infiltration of these cells in different tissues, especially lung tissue, which are causal, or at the least concomitant, phenomena, with a deterioration of the endothelial vascular wall.

It has also been established, in particular in document WO-02/39123, a correlation between the amount of circulating protein endocan and the severity of the sepsis in the patients.

It has also been demonstrated that patients suffering from non-severe sepsis had a concentration of circulating protein endocan which, although low, was significantly detectable and significantly higher than the concentration of protein endocan found in the serum or the plasma of healthy volunteers.

It has been also shown in the art that the development of the levels of C-reactive protein and of soluble ICAM-1 protein do not correlate with the development of the concentration of endocan.

In contrast, there is a good correlation between the development of the levels of procalcitonin and of protein endocan. However, the biological significance of procalcitonin in the case of sepsis is not known and this protein therefore does not represent a good biological marker for the development of sepsis.

In addition, the quantity of serum or plasma endocan found in patients suffering from sepsis represents a reliable diagnosis of mortality for these patients.

Nevertheless, there is still a need in the art for non-invasive method and new reliable biomarkers, which could be used, eventually in combination with endocan or other suitable marker(s), for determining the occurrence of an inflammatory state in a subject.

### SUMMARY OF THE INVENTION

It has been found according to the invention that circulating protein endocan is cleaved in vivo by various enzymes, including neutrophil protease cathepsin G, and that in vivo proteolysis of circulating endocan generates enzyme-resistant endocan-specific peptide fragments, thereof the amount accurately reflect the inflammatory status of the body organism.

It has thus been found according to the invention that the said endocan-specific, enzyme resistant, peptide fragments constitute novel biomarkers that are useful for determining the inflammatory state of an individual at a given period of time.

As a result, the present invention relates to an enzyme proteolysis-resistant peptide that binds to antibodies directed against the amino acid region 1-116 of the endocan's polypeptide sequence of SEQ ID N^{o}1, which peptide possesses an apparent molecular weight of 14 kDa.

The said enzyme proteolysis-resistant peptide is advantageously cathepsin G-resistant.

The endocan's peptide of SEQ ID N^{o}1 may be obtained from the full-length endocan encoded by the nucleic acid of SEQ ID N^{o}2.

In a preferred embodiment, the said peptide binds advantageously to the antibody selected from the group consisting of MEC36 and MEP21 [CNCM n^{o}I-1944].

In a preferred embodiment, the said peptide is advantageously selected from the group of peptides consisting of

(i) a peptide having a MALDI-TOF mass of 11974 Daltons,

(ii) a peptide having a MALDI-TOF mass of 12483 Daltons, and

(iii) a peptide having a MALDI-TOF mass of 12638 Daltons.

In another preferred embodiment, the said peptide is advantageously selected from the group of peptides consisting of:

(i) a peptide having the amino acid sequence 1-111 of the human endocan sequence of SEQ ID N^{o}1,

(ii) a peptide having the amino acid sequence 1-115 of the human endocan sequence of SEQ ID N^{o}1, and

(iii) a peptide having the amino acid sequence 1-116 of the human endocan sequence of SEQ ID N^{o}1.

The present invention further relates to a recombinant peptide p14, that is selected from the group consisting of:

(i) a recombinant peptide having at least 90% amino acid identity with the amino acid sequence 1-111 of the human endocan sequence of SEQ ID N^{o}1,

(ii) a recombinant peptide having at least 90% amino acid identity with the amino acid sequence 1-115 of the human endocan sequence of SEQ ID N^{o}1, and

(iii) a recombinant peptide having at least 90% amino acid identity with the amino acid sequence 1-116 of the human endocan sequence of SEQ ID N^{o}1.

The present invention also relates to a monoclonal antibody which is specific to one or more peptide p14 or recombinant p14 as defined above and which does not detectably bind to the endocan protein having the amino acid sequence of SEQ ID N^{o}1.

The present invention also relates to a method for determining the occurrence of an inflammatory state in a subject, comprising the steps of:

a) providing a sample previously collected from the said subject, advantageously a serum or a plasma sample,

b) measuring the amount value of at least one enzyme proteolysis-resistant peptide as defined above, in the said sample,

c) determining the inflammatory state of the said subject from the peptide amount value measured at step b).

In a preferred embodiment, said step b) further comprises measuring the amount value of human endocan of SEQ ID N^{o}1 in the said sample, and said step c) consists of determining the inflammatory state of the said subject from the ratio of the amount values of (i) the at least enzyme proteolysis-resistant peptide, to (ii) the human endocan of SEQ ID N^{o}1 measured at step b).

According preferred characteristics, the said inflammatory state is selected from the group consisting of:

a chronic inflammatory state and an acute inflammatory state,

sepsis, acute sepsis and septic chock, or

acute lung injury (ALI) or acute respiratory distress syndrome (ARDS).

In another particular embodiment, step b) is performed by determining the intensity value of the signal that is generated by the said peptide biomarker when the said sample is subjected to a mass spectrum analysis or an immunoassay analysis.

The present invention also concerns a method for monitoring the treatment efficiency of a patient affected with an inflammatory state, comprising a step of performing the method above-mentioned with one or more samples that have been collected from the said patient at one or more instants.

The invention further relates to a method for the in vivo testing of a candidate anti-inflammatory substance, comprising the steps of:

a) providing a sample, preferably a serum or a plasma sample, from a patient in need of an anti-inflammatory treatment to whom the said candidate substance has been administered prior to collecting the said sample,

b) performing the method for determining the occurrence of an inflammatory state in a subject that is described above on the said patient (i.e. measuring the amount value of at least one enzyme proteolysis-resistant peptide as defined above, in the sample of step (b)), and

c) determining the anti-inflammatory effect of the said candidate substance on the said patient.

In another aspect, the invention concerns a kit for determining the inflammatory state of a subject, the said kit comprising means necessary for measuring the amount value of at least one peptide above-mentioned in a sample collected from the said subject, and further advantageously comprises means necessary for measuring the amount value of human endocan of SEQ ID N^{o}1 in the said same subject's sample.

In some embodiments, the said kit also comprises a standard sample comprising one or more peptide biomarkers or recombinant peptides as defined above.

In a particular embodiment, the said kit comprises a solid support comprising at least one capture reagent attached thereto, wherein the said capture reagent binds to at least one peptide above-mentioned.

This kit further comprises

a) means for detecting the formation of complexes between a capture reagent attached to the said solid support and one of the said peptide biomarkers, or a ligand molecules that specifically bind to one of the said peptide biomarkers, and

b) instructions for using the said solid support to detect one or more of the at least one peptide biomarker.

The invention also relates to a method for the in vitro screening of anti-inflammatory candidate substances comprising the steps of:

a) providing an assay sample comprising human endocan of SEQ ID N^{o}1 and cathepsin G in the presence of the candidate substance to be tested; and

b) determining the amount value of the preceding peptide in the said assay sample.

This method further comprises advantageously a step c) of selecting positively the said candidate substance when the amount value of the above-mentioned peptide is equal to, or lesser than, a reference amount value that is expected when step a) is performed with a cathepsin G inhibitor.

### BRIEF DESCRIPTION OF THE FIGURES

FIG. 1: Involvement of PMN-derived elastase and cathepsin G in the degradation of Endocan.

(A) Supernatants from PMA-activated PMN degrade endocan in a serine protease-dependent manner. PMN supernatant was first incubated with or without inhibitors for 2 h at 37° C., and then endocan was added for a 24 h incubation at 37° C. Controls were inhibitors incubated for 24 h with endocan. Residual endocan were measured by ELISA. Results are expressed in percentage of endocan degradation [1−(E+inhibitor+PMN sup)/(E+inhibitor)]*100.

(B) Degradation profile and dose response are specific for each PMN-derived serine protease. Endocan (2 μg/ml) was incubated with elastase, cathepsin G or proteinase 3 at concentrations ranging from 0.001 μg/mL to 1 μg/mL (lanes 3 to 7). After 24h incubation at 37° C., the samples were studied by western blot with MEP 21 monoclonal anti-endocan antibody. The controls were endocan without PMN supernatants (lane 1), and endocan incubated with 10% activated PMN supernatant (lane 2), both were also incubated 24 h at 37° C.

(C) Specific inhibition of elastase and cathespin G abolished the degradation of endocan by PMN supernatants. Western blot revealed with the MEP 21 monoclonal anti-endocan antibody. Positive control: activated PMN supernatant-treated endocan (lanes 1 and 7). Negative control: inhibitor-(lane 2) or cathepsin G inhibitor-(lane 8)-treated endocan. Endocan incubated with 10% activated PMN supernatants and decreasing concentrations of elastase inhibitor (lanes 3 to 6). Endocan incubated with 10% activated-PMN supernatant and decreasing concentrations of cathepsin G inhibitor (lanes 9 to 12).

FIG. 2: Cathepsin G but not elastase generates a major endocan catabolite of 14 kDa (p14).

Kinetics of endocan degradation by activated-PMN supernatant (10% v/v), by elastase (0.1 μg/ml), or by cathepsin G (0.01 μg/ml). Western blots were revealed by MEP 21 antibody. Arrow indicates native endocan. Arrow heads indicate endocan degradation products. The endocan controls at the first lane of each immunoblot, were also incubated for 72 h at 37° C.

FIG. 3: Characterization of endocan p14.

After cleavage of endocan by cathepsin G, the mass of endocan p14 was determined by MALDI-TOF mass spectrometry.

FIG. 4: Biological activity of endocan p14.

(A) Endocan binds to Jurkat by its protein core.

10×10⁶ Jurkat cells were incubated with transfected HEK cell supernatants containing endocan or the non glycanable endocan mutant (S137A/E). After 1 h at 4° C., Jurkat cells were washed 3 times with RPMI1640 and lysed. Measurement of endocan in cell lysates has been already shown to reflect directly the quantity of bound endocan onto the surface of Jurkat cells (Bechard D et al, J Immunol, 2001). Results are expressed in pg of bound endocan per 10⁶ cells.

(B) Non glycanable endocan is co-immunoprecipitated with anti-LFA-1 mabs. Bound endocan from 20 to 40×10⁶ Jurkat cells were incubated with a mab against endocan (MEC36), mabs against LFA-1 (HI111 and mab24), and anti-CD3 mab as control antibody. Bound endocan is expressed in pg per 10⁶ cells.

(C) Endocan p14 inhibits binding of endocan to Jurkat cells. Jurkat cells (10×10⁶) were first incubated with p14, endocan's glycan, cathepsin G or the buffer supplemented with MnCl₂ and CaCl₂ for 1 h at 4° C. Then, cell supernatant-containing endocan was added. After 1 h at 4° C., Jurkat cells were washed and lysed. Bound endocan is expressed in pg per 10⁶ cells.

FIG. 5: Presence of endocan p14 in the serum from septic shock patients.

Western blot analysis of endocan and its degradation products immunoprecipitated with MEC36-agarose beads from normal serum (lane 5) and serum from septic shock patients (lane 4). The other lanes include different controls. Lane 1: untreated recombinant endocan; lane 2: cathepsin G-treated recombinant endocan; lane 3: neutrophil supernatant-treated recombinant endocan; lane 6: eluate from the MEC 36-agarose beads alone.

FIG. 6: Assay to screen inhibitors of cathepsin G—endocan interactions.

FIG. 6 shows the evolution of the final amount of endocan after incubation with cathepsin G (white circle), with cathepsin G and cathepsin G inhibitor (filled circle) or without cathepsin G (control, white diamonds) relating to its initial concentration. The final amount of endocan is measured by an immunoassay based on the use of MEC-15 antibody. The details of the experiment are given further below in Example 3. Y-coordinate: Optical density at 450 nm. X-coordinate: initial endocan concentration (i.e. before incubation).

FIG. 7: Detection of serum antibodies against p14 C-terminal fragments in pre-immune and immune serum. The pre-immune and immune serums were obtained from mice and rats immunized with KLH-peptide 1 conjugate (7A) and KLH-peptide 2 conjugate (7B). The experimental details are given further below in Example 1. Y-coordinate: Optical density unit at 450 nm. X-coordinate: serum dilution. Curve 1: Mouse 1 pre-immune serum, Curve 2 : Mouse 1 immune serum, Curve 3: Mouse 2 pre-immune serum, Curve 4: Mouse 2 immune serum, Curve 5: Rat 1 pre-immune serum, Curve 6: Rat 1 immune serum, Curve 3: Rat 2 pre-immune serum, Curve 4: Rat 2 immune serum,

FIG. 8: Dose-response inhibition of NK and YAC-1 cell transendothelial migration by endocan

FIG. 8 shows the percentage of leukocyte transendothelial migration in the presence of increasing amount of endocan. FIG. 8A refers to NK cells and FIG. 8B refers to YAC-1 cells. The experimental details are given further below in Example 3. X-coordinate: concentration of endocan in ng/ml. Y-coordinate: percentage of transendothelial migration. Statistical analysis (Kruskall-Wallis): *p<0.05, **p<0.01

FIG. 9: Competitive immunoassay

FIG. 9 shows the result of competitive binding of immune serum (from rat immunized by KLH-peptide 1 conjugate) to coated BSA-peptide in the presence of endocan (1), crude CHO-DG44 cell supernatant containing recombinant p111 (2), crude CHO-DG44 cell supernatant containing p115 (3) or crude CHO-DG44 cell supernatant containing p116. Controls are performed in the absence of endocan and p14 peptides and in the presence of crude cell supernatant (5) or buffer (6). The experimental details are given further below in Example 1. Y-coordinate: Optical density at 450 nm.

### DETAILED DESCRIPTION OF THE INVENTION

It has been found according to the present invention that there is a direct relationship between (i) the amount value of endocan-specific, enzyme-resistant peptides contained in a biological sample from an individual and (ii) the occurrence of, or the level of, an inflammatory reaction in the said individual's body.

Notably, it has been found according to the invention that although the determination of the amount of plasma or serum endocan in an individual may allow to discriminate between (i) individuals who are affected with a severe sepsis, e.g. a septic shock and (ii) individuals who are not affected with a severe sepsis, the said determination of plasma endocan does not reflect accurately or precisely the inflammatory state of the said individual, which inflammatory state includes the level of proteolysis activity of enzymes that are produced, or over-produced, during an inflammatory reaction, like for example elastase and cathepsin G.

In contrast, the invention finds that endocan-specific, enzyme-resistant peptides contained in a biological sample from an individual is in a direct relationship with the enzyme activity of proteases that are produced, or over-produced, during an inflammatory reaction occurring in the said individual's body; this finding has allowed the inventors to use the said endocan-specific peptides, as biomarkers of an inflammatory reaction.

More precisely, because the amount value of endocan-specific proteolysis-resistant peptides that is found in an individual's biological sample is linked to the activity of proteases involved in an inflammation reaction, the said amount value of endocan-specific proteolysis-resistant peptides according to the invention may be used as a biomarker of the inflammatory state of the said individual.

These results have allowed the development of tools that can be used in the determination of the occurrence of, or the level of, an inflammatory state in a subject, in particular for a subject having, or suspected to be affected with a neutrophil granulocytes inflammation disease of the acute type (sepsis, ARDS) or chronic type (chronic broncho-pneumonia, fibrosis pulmonary, emphysema).

Thus, new endocan-specific polypeptide biomarkers have been identified and characterized, which novel biomarkers are usable in methods or kits to determine the occurrence of an inflammatory state in a patient, or to determine the level of an inflammatory state in a patient, which inflammatory state encompasses acute and chronic inflammatory diseases associated with activated polymorphonuclear neutrophil leukocytes.

As shown in the examples herein, proteolysis of endocan by the neutrophil protease cathepsin G produces a major peptide degradation product of about 14 kDa, which is found notably in sera originating from septic shock patients.

In turn, this peptide degradation product inhibits in vitro the binding of endocan to the human leukocyte cell line Jurkat.

These results show that expression of cathepsin G quickly after PMN activation switches the anti-inflammatory action of endocan to a pro-inflammatory component.

This new endocan pathway may participate to the complex network that controls PMN margination, sequestration and migration towards the pulmonary capillaries.

1.—General Definition of a Biomarker

A biomarker is an organic biomolecule, the presence of which in a sample is used to determine the occurrence of a disease and in particular the phenotypic status of the subject (e.g., patient presenting an inflammatory state v. normal or non-affected patient).

In a preferred embodiment, the biomarker is differentially present in a sample collected from a subject of one phenotypic status (e.g., having a disease) as compared with another phenotypic status (e.g., not having the disease).

In another preferred embodiment, the biomarker is also quantitatively differentially present in a sample taken from a subject to another, to determine the severity of the disease (e.g., between a chronic inflammatory state and an acute inflammatory state, or from sepsis to septic chock, or between an acute lung injury (ALI) and an acute respiratory distress syndrome (ARDS)).

A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio.

Biomarkers, alone or in combination, provide measures of relative risk that a subject belongs to one phenotypic status or another. Therefore, they are useful as markers in particular for disease (diagnostics), therapeutic effectiveness of a drug (theranostics) or drug toxicity.

2.—Biomarkers According to the Invention

This invention provides, among other useful features, polypeptide-based biomarkers that are useful for determining the occurrence of an inflammatory state, in particular of chronic or acute type, in a subject.

This invention also provides the same polypeptide-based biomarkers that are useful for determining the level of an inflammatory state, in particular of chronic or acute type, in a subject.

These polypeptide biomarkers are differentially present in subjects presenting an inflammatory state (in particular septic shock patients) versus healthy individuals.

These polypeptide biomarkers are also quantitatively present in a biological sample from the subject, especially in a serum or a plasma sample, depending on the severity of the inflammatory state. In particular, these biomarkers can potentially be used to discriminate between sepsis, acute sepsis and septic chock.

Clinical proteomic is a validated approach that has enabled the determination of the new serum biomarkers in samples of subjects presenting an inflammatory state.

As shown in the examples herein, the degradation product of endocan that is obtained after proteolysis by cathepsin G consists of a small family of endocan-specific peptides that are resistant to further enzyme proteolysis, including to further enzyme proteolysis by cathepsin G.

Said small family of endocan-specific peptides, and in particular each peptide of said family, has an apparent molecular weight of about 14 kDa, notably as determined by Western blotting.

Further, each of the said endocan-specific peptides may be individually characterized or purified, such that each of the said endocan-specific peptides consists of a peptide biomarker according to the present invention.

One object of the invention consists of an enzyme proteolysis-resistant peptide that binds to antibodies directed against the amino acid region 1-116 of the endocan's polypeptide sequence of SEQ ID N^{o}1, which peptide possesses an apparent molecular weight of 14 kDa.

The endocan-specific peptide biomarkers according to the invention may collectively be interchangeably termed “p14”, “p14 biomarkers” or “endocan p14” herein. Thus, “p14” encompasses both (i) the small family of proteolysis-resistant endocan-specific peptides that are generated by cathepsin G proteolysis of endocan and (ii) each of the individual peptides comprised in the said small family of peptides which have an apparent molecular weight of about 14 kDa.

As used herein, the expression “proteolysis-resistant”, as applied to the peptide biomarkers of the invention, means that the said peptide biomarkers are not further cleaved by cathepsin G or another protease of human polynuclear neutrophil origin.

Preferably, the expression “proteolysis-resistant”, as applied to the peptide biomarkers of the invention, means that the said peptide biomarkers are not further cleaved by cathepsin G or elastase.

Because the p14 biomarkers according to the invention are characterized by their proteolysis-resistance, binding properties and apparent molecular weight, they can easily be detected without requiring the knowledge of their specific identity.

In particular, the kinetics of degradation by cathepsin G showed that this protease cleaved endocan in a single and stable fragment having a apparent molecular weight of 14 kDa, from 2 to 72 hours incubation. This endocan p14, even in presence of elastase, remained present all over the time course of the kinetics.

The p14 biomarkers are able to bind to antibodies selected from the group consisting of (i) antibodies that are specifically directed to the antigenic determinant AgD1 of mature endocan, i.e. the 60-80 amino acid region of SEQ ID N^{o}1 and (ii) antibodies that are specifically directed to the N-terminal region of mature endocan, i.e. the 105-116 amino acid region of SEQ ID N^{o}1.

As shown in the examples herein, the p14 biomarkers are able to bind notably to antibodies MEC 36 or MEP 21 (hybridoma cell line CNCM 1-1944). The antibodies termed MEP 21 consist of the monoclonal antibodies that are produced by the hybridoma cell line deposited on 19 Nov. 1997 at the Collection Nationale de Cultures de Microorganismes from Institut Pasteur (Paris) under the accession number 1-1944.

The p14 biomarker's apparent molecular weight is preferably determined by a Western blotting assay under reducing conditions.

The p14 biomarkers of this invention are further characterized by their mass-to-charge ratio as determined by mass spectrometry, by the shape of their spectral peak in time-of-flight mass spectrometry.

These characteristics provide one method to determine whether a specific detected biomolecule consists of a p14 biomarker of this invention. These characteristics represent inherent characteristics of the p14 biomarkers and do not introduce process limitations in the manner in which the biomolecules are discriminated.

The p14 biomarkers were also identified herein using MALDI technology, e.g. MALDI-TOF Voyager Elite mass spectrometry (Perspective Biosystem, Framingham, Mass., USA).

This method is described in more detail in the Example Section.

The p14 biomarkers thus encompass, or consist of, the following ones:
(i) a peptide having a MALDI-TOF mass of 11974 Daltons,
(ii) a peptide having a MALDI-TOF mass of 12483 Daltons, and
(iii) a peptide having a MALDI-TOF mass of 12638 Daltons.

Moreover, the p14 biomarkers of this invention may further be characterized by the shape of their spectral peak in time-of-flight mass spectrometry. Mass spectra showing peaks representing the biomarkers are presented in FIG. 3B.

The p14 biomarkers may also be characterized by their amino acid sequences.

More specifically, the p14 biomarkers encompass the following endocan-specific proteolysis-resistant peptides:
(i) a peptide having the amino acid sequence 1-111 of the human endocan sequence of SEQ ID N^{o}1,
(ii) a peptide having the amino acid sequence 1-115 of the human endocan sequence of SEQ ID N^{o}1, and
(iii) a peptide having the amino acid sequence 1-116 of the human endocan sequence of SEQ ID N^{o}1.

The p14 biomarkers (i), (ii) and (iii) are also termed herein by p111 peptide, p115 peptide and p116 peptide, respectively.

The p14 biomarkers identity can be also characterized by determining the amino acid sequence of the polypeptides.

For example, a biomarker can be peptide-mapped with a number of enzymes, such as trypsin (“trypsic fingerprinting”), and the molecular weights of the digestion fragments can be used to compare with non glycanable endocan S137A/E (see Table 1 at the end of the specification).

Alternatively, p14 biomarkers may be sequenced using tandem MS technology. In this method, the protein is isolated, for example by gel electrophoresis. A band containing the biomarker is cut out and the protein is subject to protease digestion. Individual protein fragments are separated by a first mass spectrometer. The fragment is then subjected to collision-induced cooling, which fragments the peptide and produces a polypeptide ladder. A polypeptide ladder is then analyzed by the second mass spectrometer of the tandem MS. The difference in masses of the members of the polypeptide ladder identifies the amino acids in the sequence. An entire protein can be sequenced this way, or a sequence fragment can be subjected to database mining to find identity candidates.

The preferred biological source for detection of the p14 biomarkers is serum.

This invention provides the p14 biomarkers in a purified or in an isolated form. The p14 biomarkers can be purified or isolated from biological fluids, such as from human serum samples. The p14 biomarkers can be isolated by any method known in the art, based on both their mass and their binding characteristics.

For example, a sample comprising a p14 biomarkers may be subject to chromatographic fractionation, as described herein, and subject to further separation by, e.g. acrylamide gel electrophoresis.

Knowledge of the identity of the p14 biomarkers according to the present invention also allows their purification or isolation from a starting biological sample by immunoaffinity separation techniques, as it is shown in the examples herein.

The p14 biomarkers may also be produced by enzyme degradation of endocan of SEQ ID N^{o}1, e.g. by incubation of a purified endocan with activated-PMN cell culture supernatant or by incubation of a purified endocan in a cathepsin G-containing solution, as disclosed in the examples herein.

3.—Detection of Biomarkers for Determining the Occurrence of an Inflammatory State in a Subject

The biomarkers of this invention can be detected by any suitable method.

Detection methods that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

Protein biochips are biochips adapted for the capture of polypeptides. Many protein biochips are described in the art. These include, for example, protein biochips produced by Ciphergen Biosystems, Inc. (Fremont, Calif.), Packard BioScience Company (Meriden Conn.), Zyomyx (Hayward, Calif.), Phylos (Lexington, Mass.) and Biacore (Uppsala, Sweden). Examples of such protein biochips are described in the following patents or published patent applications: U.S. Pat. No. 6,225,047; PCT International Publication No. WO 99/51773; U.S. Pat. No. 6,329,209, PCT International Publication No. WO 00/56934 and U.S. Pat. No. 5,242,828.

3.1.—Detection by Mass Spectrometry

3.1.1.—Mass Spectrometry System

In a preferred embodiment, the biomarkers of this invention are detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these.

In a further preferred method, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer.

A preferred mass spectrometric technique for use in the invention is “Surface Enhanced Laser Desorption and Ionization” or “SELDI,” as described, for example, in U.S. Pat. No. 5,719,060 and U.S. Pat. No. 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe. There are several versions of SELDI.

In another mass spectrometry method, the biomarkers can be first captured on a chromatographic resin having chromatographic properties that bind the biomarkers. In the present example, this could include a variety of methods. For example, one could capture the biomarkers on a cation exchange resin, such as CM Ceramic HyperD F resin, wash the resin, elute the biomarkers and detect by MALDI (“Matrix Assisted Laser Desorption Ionisation D). Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI.

3.1.2.—Analysis

Analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip(R) software, data processing typically includes TOF-to-M/Z transformation to generate a mass spectrum, baseline substraction to eliminate instrument offsets and high frequency noise filtering to reduce high frequency noise.

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set at zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Analysis generally involves the identification of peaks in the spectrum that represent signal from an analyte. Peak selection can be done visually, but software is available, as part of Ciphergen's ProteinChip(R) software package, that can automate the detection of peaks. In general, this software functions by identifying signals having a signal-to-noise ratio above a selected threshold and labeling the mass of the peak at the centroid of the peak signal. In one useful application, many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Software used to analyze the data can include code that applies an algorithm to the analysis of the signal to determine whether the signal represents a peak in a signal that corresponds to a biomarker according to the present invention. The software also can subject the data regarding observed biomarker peaks to classification tree or ANN analysis, to determine whether a biomarker peak or combination of biomarker peaks is present that indicates the status of the particular clinical parameter under examination. Analysis of the data may be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

3.2.—Detection by Immunoassay

3.2.1—Immunoassay Technology

In another embodiment, the biomarkers of this invention can be measured by immunoassay. Immunoassay requires biospecific capture reagents, such as antibodies, to capture the biomarkers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, sandwich immunoassays including ELISA or fluorescence-based immunoassays, as well as other enzyme immunoassays.

In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

In particular, it could be used suitable antibodies such as described in documents FR-2 775 691 or WO-02/39123, and in particular anti-endocan mAb MEC 36 or MEP 21 HRP-conjugated monoclonal antibody.

The antibodies for use in the methods of the present invention, in particular any antibody that specifically binds to p14 biomarkers, can be produced using any antibody production method known to those of skill in the art.

Preferably the antibody is monoclonal in nature. By “monoclonal antibody” is intended an antibody obtained from a population of substantially homogeneous antibodies, i.e., the individual antibodies comprising the population are identical except for possible naturally occurring mutations that may be present in minor amounts. The term is not limited regarding the species or source of the antibody. The term encompasses whole immunoglobulins as well as fragments such as Fab, F(ab′)2, Fv, and others which retain the antigen binding function of the antibody. Monoclonal antibodies are highly specific, being directed against a single antigenic site; for example, in the case of anti-p14 antibodies, the C-terminal peptides of p14 biomarkers.

The term “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in, for example, Clackson et al. (1991) Nature 352:624-628; Marks et al. (1991) J Mol. Biol. 222:581-597; and U.S. Pat. No. 5,514,548.

Monoclonal antibodies can be prepared using the method of Kohler et al. (1975) Nature 256:495-496, or a modification thereof. Typically, a mouse is immunized with a solution containing an antigen. Immunization can be performed by mixing or emulsifying the antigen-containing solution in saline, preferably in an adjuvant such as Freund's complete adjuvant, and injecting the mixture or emulsion parenterally. Any method of immunization known in the art may be used to obtain the monoclonal antibodies of the invention. After immunization of the animal, the spleen (and optionally, several large lymph nodes) are removed and dissociated into single cells. The spleen cells may be screened by applying a cell suspension to a plate or well coated with the antigen of interest. The B cells expressing membrane bound immunoglobulin specific for the antigen bind to the plate and are not rinsed away. Resulting B cells, or all dissociated spleen cells, are then induced to fuse with myeloma cells to form hybridomas, and are cultured in a selective medium. The resulting cells are plated by serial dilution and are assayed for the production of antibodies that specifically bind the antigen of interest (and that do not bind to unrelated antigens). The selected monoclonal antibody (mAb)-secreting hybridomas are then cultured either in vitro (e.g., in tissue culture flasks, bottles or hollow fiber reactors), or in vivo (as ascites in mice).

Also in a preferred embodiment, it is used monoclonal antibodies which are specific to the neo-antigenic determinant formed by the C-terminal extremity of polypeptide p14.

These monoclonal antibodies specific to C-terminal extremity can be obtained for instance by the following general protocol:

Construction of C-terminal peptides (8 to 25 amino acids) of p14 biomarkers, then bound said C-terminal peptides to a suitable carrier protein,

Immunization in foot pad and subcutaneously of a balb/c mouse and a Lewis rat with the C-terminal peptides immobilized to the carrier protein in complete Freund adjuvant, and then followed by 4 to 5 antigen boosts; at the end of immunization, lymphocytes from the spleen and the regional lymph node are purified and then fused with Sp2/0 myeloma cells accordingly with the published protocol from Kohler et al. (1975) Nature 256:495,

the fused cells are cultured in microplates, and 10 to 14 days later, selection of the hybridoma cells lines which product monoclonal antibodies specific to said C-terminal peptides could occur.

The present invention also relates to a monoclonal or a polyclonal antibody specific to one or more p14 peptides.

As intended herein, one or more p14 peptides include anyone of p14 peptides, two of the p14 peptides and the three p14 peptides.

Preferably, the said monoclonal or polyclonal antibody does not detectably bind to endocan of SEQ ID N^{o}1.

In preferred embodiments, the said monoclonal or polyclonal antibody has low or no affinity to native endocan. The affinity of the said monoclonal for human endocan of SEQ ID N^{o}1 and for p14 peptides can be assessed by well-known methods of the prior art. For example, the one skilled in the art may determine the dissociation constant (Kd) using Surface Plasmon Resonance (SPR) experiments. For this purpose the p14 peptides and the human endocan of SEQ ID N^{o}1 may be immobilized on different sensor chips for SPR.

A monoclonal or polyclonal antibody has low or no affinity to native endocan and is specific to a p14 peptide if its Kd for a p14-peptide is at least 10-fold more preferably 100-fold lower than that its Kd for endocan. Preferably, the Kd for at least one p14 peptide is lower than 1 mM.

As an alternative, the one skilled in the art may perform an immunoassay as described in example 1 of the present specification (see section entitled Evaluation of the specificity of polyclonal antibodies in Material and Methods of Example 1).

The said immunoassay enables to compare the bond strength of the antibody for endocan and for one of the p14 peptide. The specific signals detected for endocan and the p14 peptide of interest are compared. The antibody is specific for p14 peptides if its specific signal in the presence of one of the p14 peptide is significantly different (in term of value) from its specific signal in the presence of endocan.

In a preferred embodiment, the said antibody which is specific to one or more p14 peptides and which does not detectably bind endocan of SEQ ID N^{o}1 is a monoclonal antibody.

3.2.2—General Protocol of Immunoassay for Detection of Biomarkers for Determining the Occurrence of an Inflammatory State

A preferred protocol for the detection by immunoassay of the biomarkers of this invention is as follows.

The detection will be performed on serum or plasma sample, from a patient to diagnose. The sera is cleared by centrifugation, followed by filtration, and then diluted in medium containing anti-protease mix.

For immunoprecipitation, a first type of monoclonal antibodies is coupled to an agarose support matrix, e.g. MEC 36 or MEP 21 (hybridoma cell line CNCM I-1944). Monoclonal antibodies-agarose beads are added to the sera. Agarose beads are collected by centrifugation, washed.

After centrifugation, the beads are resuspended in SDS-PAGE sample buffer, DTT added and samples are studied by western blot with another monoclonal antibody, e.g. MEC 36 or MEP 21 (hybridoma cell line CNCM I-1944).

4.—Determination of the Occurrence of, or the Level of, an Inflammatory State in a Subject

4.1.—Single Markers

As it is extensively described above in the present specification, p14 biomarkers of the invention can be used in diagnostic tests to determine the occurrence of inflammatory state in a subject, e.g. a chronic inflammatory state and an acute inflammatory state.

The expression “inflammatory state” relates in particular to inflammation due to polymorphonuclear neutrophils, and includes distinguishing, inter alia, subject having inflammation v. subject not having such an inflammation. They also relate to group consisting of a chronic inflammatory state and an acute inflammatory state, or to group consisting of.

The diagnostic test includes also determining the severity of the inflammatory state, and in particular distinguishing between sepsis, acute sepsis and septic chock, or between acute lung injury (ALI) and acute respiratory distress syndrome (ARDS).

Based on this status, further procedures may be indicated, including additional diagnostic tests or therapeutic procedures or regimens.

The power of a diagnostic test to correctly predict the occurrence disease is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of actual positives that test as positive. Negative predictive value is the percentage of actual negatives that test as negative.

Each of the biomarkers described herein is individually useful for determining the occurrence of an inflammatory disease.

The method involves, first, providing a sample previously collected from the subject, second, measuring at least one the above-mentioned biomarkers in the said sample using at least one of the methods described herein, third, determining the occurrence of an inflammation state from the biomarker values measured in second step.

The values measured represent a measured amount of a biomarker which allows determining in particular the inflammation status of the tested subject.

In a preferred embodiment of the present invention, the occurrence and the severity of the inflammation state is advantageously determined by measuring the amount of at least one of the biomarkers described herein. Specifically, the increase of the severity of the inflammation state is correlated to the increase of the amount of biomarkers in accordance to the invention.

The amount of the at least one biomarker is advantageously determined in accordance of at least one of the methods described herein.

The measured amount is then submitted to a classification algorithm or compared to a reference amount and/or pattern of biomarkers that is associated with the particular state of the disease.

Also in a preferred embodiment, the severity of the inflammation state is determined by performing the following steps: first, determining the intensity value of the signal that is generated by the said biomarker when the said sample is subjected to a suitable analysis method, second, comparing the signal intensity value obtained at first step with at least a reference signal intensity value that is expected to be measured in an individual selected form the group consisting of (i) an individual who is not affected by a inflammatory disease, (ii) an individual who is affected with sepsis, (iii) an individual who is affected with an acute sepsis, and (iv) an individual who is affected with a septic shock, and third, determining the severity of the inflammatory state of the tested subject.

4.2.—Combination of Markers

While individual biomarkers are useful diagnostic biomarkers, a combination of markers can also provide greater predictive value of a particular status. Specifically, the detection of a plurality of biomarkers in a sample can increase the sensitivity and/or specificity of the test.

It has been found in vivo that during septic shock syndrome, two forms of endocan protein are detected in serum or plasma of patient: (i) the native endocan and (ii) the p14 biomarkers of the invention (which represent a specific product of cathepsin G derived from PMN).

In a preferred embodiment, the severity of the inflammatory state may be thus determined in a third step as being function of the ratio of the amount values of (i) the at least one peptide p14 biomarker defined herein, to (ii) the human endocan of SEQ ID N^{o}1 measured in the same patient sample.

In practice, the amount value of the human endocan of SEQ ID N^{o}1 is advantageously measured in the same sample as p14 biomarkers.

This amount value of the human endocan of SEQ ID N^{o}1 can be measured by mass spectrometry method or immunoassay. The measurement method of endocan is advantageously as disclosed in particular in document documents FR-2 775 691 and WO-02/39123.

In other embodiments of the invention, at least one of the following prognostic markers, known in the art, can also be used in combination with p14 biomarkers of the invention, i.e. for example and not limited to, serum interleukin (IL)-18, soluble intercellular adhesion molecule-1 (ICAM-1), the cellular expression of cell adhesion molecules like ICAM-I and CD40, serum soluble IL-2 receptor (sIL-2R) levels, intestinal multidrug resistance protein (MDRI), serum leucine aminopeptidase, C-reactive protein, procalcitonin, elastase, cathepsin G and/or alpha 1-antitrypsin.

4.3.—Subject Management

In certain embodiments of the methods of determining the occurrence of inflammation state, the methods further comprise managing subject treatment based on the status. Such management includes the actions of the physician or clinician subsequent to determining inflammatory state, using in particular the p14 biomarkers of the invention.

For example, if a physician makes a diagnosis of inflammatory state by using notably the biomarkers of the invention, then a certain regime of treatment, such as prescription or administration of anti-inflammatory medicaments might follow.

Then, when the physician makes a diagnosis of inflammatory state in a subject, the p14 biomarkers, alone or with associated useful biomarkers, allow to monitoring the treatment efficiency.

This treatment, to be effective, may result in a reduction of the p14 biomarkers amount value (or alternatively the decrease of the p14/endocan ratio) in the serum or plasma sample of the patient.

In another embodiment, the p14 biomarkers can also be advantageously efficient in a method for the in vivo testing of a candidate anti-inflammatory substance, as disclosed in details herein under.

This method consists advantageously in, first, administering the said candidate substance to a patient in need of an anti-inflammatory medical treatment, second, performing the method for measuring the amount value of at least one peptide marker of the invention on the said patient, and third, determining the anti-inflammatory effect of the said candidate substance on the said patient.

5.—Recombinant p14 Peptides

The present invention also relates to a recombinant p14 peptide, the said recombinant peptide is selected from the group consisting of:

(i) a recombinant peptide having at least 90% amino acid identity with the amino acid sequence 1-111 of the human endocan sequence of SEQ ID N^{o}1,

(ii) a recombinant peptide having at least 90% amino acid identity with the amino acid sequence 1-115 of the human endocan sequence of SEQ ID N^{o}1, and

(iii) a recombinant peptide having at least 90% amino acid identity with the amino acid sequence 1-116 of the human endocan sequence of SEQ ID N^{o}1

As intended herein, a determined polypeptide having at least about 90% amino acid identity with a reference polypeptide possesses at least about 90%, 91%, 92%, 93%, 94%, 95%, 96%, 97%, 98%, 99% or 99.5% amino acid identity with the said reference polypeptide. At least about 90% amino acid identity also includes 100% amino acid identity with the reference peptide.

To determine the percent of identity of two amino acid sequences, the sequences are aligned for optimal comparison purposes. For example, gaps can be introduced in one or both of a first and a second amino acid sequence for optimal alignment and non-homologous sequences can be disregarded for comparison purposes. For optimal comparison purposes, the percent of identity of two amino acid sequences can be achieved with CLUSTAL W (version 1.82) with the following parameters: (1) CPU MODE=ClustalW mp; (2) ALIGNMENT= [symbol] full [symbol] ; (3) OUTPUT FORMAT= [symbol] aln w/numbers [symbol] ; (4) OUTPUT ORDER= [symbol] aligned [symbol] ; (5) COLOR ALIGNMENT= [symbol] no [symbol] ; (6) KTUP (word size)= [symbol] default [symbol] ; (7) WINDOW LENGTH= [symbol] default [symbol] ; (8) SCORE TYPE= [symbol] percent [symbol] ; (9) TOPDIAG= [symbol] default [symbol] ; (10) PAIRGAP= [symbol] default [symbol] ; (11) PHYLOGENETIC TREE/TREE TYPE= [symbol] none [symbol] ; (12) MATRIX= [symbol] default [symbol] ; (13) GAP OPEN= [symbol] default [symbol] ; (14) END GAPS= [symbol] default [symbol] ; (15) GAP EXTENSION= [symbol] default [symbol] ; (16) GAP DISTANCES= [symbol] default [symbol] ; (17) TREE TYPE= [symbol] cladogram [symbol] et (18) TREE GRAP DISTANCES= [symbol] hide [symbol] .

In a preferred embodiment, the said recombinant p14 peptide is selected from the group consisting of:

(i) a recombinant peptide having the amino acid sequence 1-111 of the human endocan sequence of SEQ ID N^{o}1,

(ii) a recombinant peptide having the amino acid sequence 1-115 of the human endocan sequence of SEQ ID N^{o}1, and

(iii) a recombinant peptide having the amino acid sequence 1-116 of the human endocan sequence of SEQ ID N^{o}1

Herein, the recombinant peptide (i), (ii) and (iii) are also termed by recombinant p111, recombinant p115 and recombinant p116.

A nucleic acid encoding for a p14 peptide can be obtained by a variety of methods known in the prior art. For example, a nucleic acid encoding for a p14 peptide can be obtained from endocan cDNA by site-directed mutagenesis (as illustrated herein in Example 1) or by conventional peptide synthesis.

Once a nucleic acid is provided, the corresponding recombinant p14 peptide can be obtained by any method known in the art. The said nucleic acid may be incorporated into an expression vector. Expression vectors typically include the nucleic acid encoding for the p14 peptide of interest which is operably linked to control or regulatory sequences, selectable markers, any fusion partners, and/or additional elements. The recombinant p14 peptide may be produced by culturing a host cell—transformed with an expression vector containing the nucleic acid encoding the said peptide—under the appropriate conditions to induce or cause expression of the said peptide. A wide variety of appropriate host cell lines may be used, including, but not limited to, mammalian cells, bacteria, insect cells, and yeast.

The recombinant p111, p115 and p116 peptides may be then isolated following conventional purification methods from the resulting stably transformed cell culture. In some embodiment, the recombinant peptide can be produced under the form of a polypeptide fused to a peptide tag (for example a polyhistidine tag) in order to facilitate the purification step. The peptide tag can be removed in a subsequent step by conventional methods.

6.—Kits for Detection of Biomarkers for Determining the Occurrence of Inflammatory State

In another aspect, the present invention provides kits for qualifying/determining the inflammatory status of the patient, which kits are used to detect and/or quantify p14 biomarkers according to the invention, and advantageously also human endocan of SEQ ID N^{o}1 in the said subject's sample.

These kits allow to (1) measure the presence of cathepsin G activity in a sample (in particular a biologic sample of a patient), and (2) measure indirectly the neutrophil activity (since cathepsin G is specific to neutrophil cells).

In one embodiment, the kit comprises a solid support, such as a chip, a microtiter plate or a bead or resin having a capture reagent attached thereon, wherein the capture reagent binds p14 biomarkers of the invention, and eventually also of endocan of SEQ ID N^{o}1.

Thus, for example, the kits of the present invention can comprise mass spectrometry probes for SELDI such as ProteinChip® arrays. In the case of biospecific capture reagents, the kit can comprise a solid support with a reactive surface, and a container comprising the biospecific capture reagent.

The kit can also comprise a washing solution or instructions for making a washing solution, in which the combination of the capture reagent and the washing solution allows capture of the biomarker or biomarkers on the solid support for subsequent detection by, e.g., mass spectrometry. The kit may include more than type of adsorbent, each present on a different solid support.

The kit may also comprise means for detecting the formation of complexes between a capture reagent attached to the said solid support and one of the said p14 biomarkers, or a ligand molecule that specifically bind to one of the said p14 biomarkers.

In a further embodiment, such a kit can comprise instructions for suitable operational parameters in the form of a label or separate insert. For example, the instructions may inform a consumer about how to collect the sample, how to wash the probe or the particular biomarkers to be detected.

In yet another embodiment, the kit can comprise one or more containers with biomarker samples, to be used as standard(s) for calibration.

For example, in case of antibody-based kits, the kit can comprise, for example: (1) a first antibody (e.g., attached to a solid support) that binds specifically to the p14 biomarkers of interest, and optionally, (2) a second, different antibody that binds to the 14 biomarkers or the first antibody and is conjugated to a detectable agent. The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples that can be assayed and compared to the test sample contained. Each component of the kit is usually enclosed within an individual container, and all of the various containers are within a single package along with instructions for observing whether the tested subject is a candidate for treatment with anti-inflammatory therapeutic agent.

Any means for specifically identifying and quantifying the p14 biomarkers, in the biological sample of a candidate subject is contemplated.

Thus, in some embodiments, expression level of the p14 biomarkers of interest in a biological sample is detected by means of a binding protein, forming said “capture reagent” or “ligand molecule” above-mentioned, capable of interacting specifically with that p14 biomarkers.

Preferably, labeled antibodies, binding portions thereof, or other binding partners, may be used. The word “label” when used herein refers to a detectable compound or composition that is conjugated directly or indirectly to the antibody so as to generate a “labeled” antibody. The label may be detectable by itself (e.g., radioisotope labels or fluorescent labels) or, in the case of an enzymatic label, may catalyze chemical alteration of a substrate compound or composition that is detectable. The antibodies for detection of p14 biomarkers may be advantageously monoclonal, or may be synthetically or recombinantly produced. The amount of complexed protein, for example, the amount of biomarker protein associated with the binding protein, for example, an antibody that specifically binds to the p14 biomarkers, is determined using standard protein detection methodologies known to those of skill in the art. A detailed review of immunological assay design, theory and protocols can be found in numerous texts in the art (see, for example, Ausubel et ah, eds. (1995) Current Protocols in Molecular Biology; Greene Publishing and Wiley-Interscience, NY; Coligan et al., eds. (1994) Current Protocols in Immunology (John Wiley & Sons, Inc., New York, N.Y.)).

A variety of assays are available for detecting proteins with labeled antibodies. In a one-step assay, the target protein of interest to be detected, if it is present, is immobilized and incubated with a labeled antibody. The labeled antibody binds to the immobilized target p14 biomarker. After washing to remove unbound molecules, the sample is assayed for the presence of the label, a single protein is assayed per sample. Using newer multiplex technologies, multiple proteins can be assayed in a single sample by using different labels for each detecting antibody.

In a two-step assay, the immobilized target protein molecule of interest is incubated with an unlabeled antibody. The target protein-unlabeled antibody complex, if present, is then bound to a second, labeled antibody that is specific for the unlabeled antibody. The sample is washed and assayed for the presence of the label.

The choice of marker used to label the antibodies will vary depending upon the application. However, the choice of the marker is readily determinable to one skilled in the art. These labeled antibodies may be used in immunoassays as well as in histological applications to detect the presence of any biomarker or protein of interest. The labeled antibodies may be advantageously monoclonal. Further, the antibodies for use in detecting a protein of interest may be labeled with a radioactive atom, an enzyme, a chromophoric or fluorescent moiety, or a colorimetric tag. The choice of tagging label also will depend on the detection limitations desired. Radionuclides that can serve as detectable labels include, for example, I-131, 1-123, 1-125, Y-90, Re-188, Re-186, At-211, Cu-67, Bi-212, and Pd-109. Examples of enzymes that can serve as detectable labels include, but are not limited to, horseradish peroxidase, alkaline phosphatase, beta-galactosidase, and glucose-6-phosphate dehydrogenase. Chromophoric moieties include, but are not limited to, fluorescein and rhodamine. The antibodies may be conjugated to these labels by methods known in the art. For example, enzymes and chromophoric molecules may be conjugated to the antibodies by means of coupling agents, such as dialdehydes, carbodiimides, dimaleimides, and the like. Alternatively, conjugation may occur through a ligand-receptor pair. Examples of suitable ligand-receptor pairs are biotin-avidin or biotin-streptavidin, and antibody-antigen.

For any given protein detection assay, the biological sample, or a subsample thereof comprising the p14 biomarkers, is contacted with the binding partner, for example, the antibody, or detectably labeled antibody, for the p14 biomarkers, for a time sufficient to permit the formation of antibody-antigen complexes, and then antibody binding is detected, for example, by any means noted herein above. Antibodies, or detectably labeled forms of these antibodies, can be generated using antibody production methods well known in the art.

For example, the kit can advantageously comprise two antibody types: one directed to C-terminal end of endocan p14 and the other directed to N-terminal end of endocan p14. This kit can also comprise a standard consisting in purified endocan p14.

In further embodiments, the kit comprises a antibody which is specific to at least one p14 peptide and does not detectably bind to human endocan of SEQ ID N^{o}1.

In a preferred embodiment, the said antibody is a monoclonal antibody.

In other embodiments, the kit comprises a standard sample selected from the group to consisting of (i) a peptide having the amino acid sequence 1-111 of the human endocan sequence of SEQ ID N^{o}1, (ii) a peptide having the amino acid sequence 1-115 of the human endocan sequence of SEQ ID N^{o}1, (iii) a peptide having the amino acid sequence 1-116 of the human endocan sequence of SEQ ID N^{o}1 and (iv) their mixtures.

6. Use of Biomarkers for Determining the Occurrence of Inflammatory State in Screening Assays

The methods of the present invention have other applications as well. For example, the biomarkers can be used to screen for compounds that decrease the expression of the biomarkers in vitro and/or in vivo.

This inhibition can be advantageously an indirect mean for studying the interaction inhibition between cathepsin G and endocan. The decrease of peptide p14 level can decrease its competition action vis-à-vis native endocan. This interaction inhibition between cathepsin G and endocan can optimize the anti-inflammatory action of endocan.

In another embodiment, the biomarkers can be used to monitor the response to treatments for inflammatory state.

For example, the administration of the anti-inflammatory compound which decrease the p14 biomarkers amount value (or reduce the ratio p14/native endocan) in a biological sample of the treated patient, may demonstrate the efficiency of the anti-inflammatory treatment.

By “anti-inflammatory treatment” is intended a reduction or prevention of inflammation of the subject. Therapy with at least anti-inflammatory therapeutic agent causes a physiological response that is beneficial with respect to treatment of inflammatory disease, where the diseases involve by neutrophil granulocytes.

In an aspect, the invention provides a method for identifying compounds useful, termed also “candidate drug” or “test compound”, for the treatment of inflammatory state, which are associated with increased levels of the p14 biomarkers.

A “candidate drug” refers to any compound or molecular entity or substance whose efficacy can be evaluated using the biomarkers and the methods of the present invention. Such compounds or drugs include, e.g., chemical compounds, pharmaceuticals, antibodies, polypeptides, peptides, including soluble receptors, polynucleotides, and polynucleotide analogs, DNA, RNA, siRNA, or mixtures or chimeric molecules comprising one or more of these compounds or drugs. Many organizations (e.g., the National Institutes of Health, pharmaceutical and chemical corporations) have large libraries of chemical or biological compounds from natural or synthetic processes, or fermentation broths or extracts. Such compounds can be employed in the practice of the present invention.

At the clinical level, screening a test compound includes obtaining samples from test subjects before and after the subjects have been exposed to a test compound. The levels in the samples of one or more of the p14 biomarkers may be measured and analyzed to determine whether the levels of the biomarkers change after exposure to a test compound.

For example, the administration of the test compound which decrease the p14 biomarkers amount value (or reduce the ratio p14/native endocan) in a biological sample of the treated patient, may be a potentially efficient anti-inflammatory compound.

Subjects who have been treated with test compounds will be routinely examined for any physiological effects which may result from the treatment. In particular, the test compounds will be evaluated for their ability to decrease disease symptoms or side effects in a subject.

Alternatively, if the test compounds are administered to subjects who have previously been diagnosed with inflammatory state, test compounds will be screened for their ability to slow or stop the progression of the disease.

In yet another embodiment, the invention provides a method for treating or reducing the progression or likelihood of the inflammatory state.

The samples may be analyzed by any appropriate means as disclosed herein above.

7. Use of Biomarkers for Screening Inhibitors of Cathepsin G—Endocan Interactions

The present invention also relates to a simple immunoassay to select compounds or candidate drugs that inhibits specific cleavage of endocan by cathepsin G.

These compounds or candidate drugs selected can have potential medical application, as anti-inflammatory compounds, in particular in diseases caused by polymorphonuclear neutrophils leukocytes.

Here also, a “candidate drug” refers to any compound or molecular entity or substance whose efficacy can be evaluated using the biomarkers and the methods of the present invention. Such compounds or drugs include, e.g., chemical compounds, pharmaceuticals, antibodies, polypeptides, peptides, including soluble receptors, polynucleotides, and polynucleotide analogs, DNA, RNA, siRNA, or mixtures or chimeric molecules comprising one or more of these compounds or drugs. Many organizations (e.g., the National Institutes of Health, pharmaceutical and chemical corporations) have large libraries of chemical or biological compounds from natural or synthetic processes, or fermentation broths or extracts. Such compounds can be employed in the practice of the present invention.

The assay is based on an enzyme linked immunoassay. It comprises advantageously the main following steps: first, preincubation of candidate inhibitor compound with cathepsin G, second, incubation of both said candidate inhibitor compound/cathepsin G with bound endocan onto microtiter plates, third, washings and detection of endocan p14 (or full length endocan, non cleaved, alternatively or complementary) with a method as described herein above.

A preferred embodiment of this assay is disclosed in the following examples.

This assay could be used to screen a large panel of compounds, depending on its ability to be adapted to high throughput systems.

The invention will be described in greater detail by way of specific examples. The following examples are offered for illustrative purposes, and are not intended to limit the invention in any manner. Those of skill in the art will readily recognize a variety of non-critical parameters that can be changed or modified to yield essentially the same results.

### EXAMPLES

### Example 1

### Characterization and Production of p14 Peptides by Proteolysis, Production of Recombinant p14 Peptides, Method of Detection and Quantification of p14 Peptides, Antidodies Against p14 Peptides

A. Materials and Methods

Production and Purification of Recombinant Endocan and Non Glycanable Endocan Mutant

Endocan and its non glycanable mutant (S137A/endocan) were produced by established 293 cell lines cultured in suspension in medium without FCS (293-SFM, Gibco). Purification was conducted in 2 steps including anion exchange and affinity chromatography, as previously described in Bechard D et al (J Biol Chem. 2001; 276:48341-48349). S137A/endocan was purified in one-step affinity chromatography.

Endocan's Glycan Purification

Purified endocan's glycan was obtained by β-elimination of the bond between the proteinic structure and the dermatan sulfate glycan of endocan. Endocan was incubated with NaOH 100 mM, NaBH4 2M, 1 μl of red phenol and glacial acetic acid. After 24 h at 37° C., reaction was stopped by adding of concentrated NaOH to bring the pH to 7. Dermatan sulfate chains of endocan were then purified on DEAE-Sephacel resin (Sigma). After a contact of 30 minutes, the resin was rinsed with 10 volums of Tris 50 mM, NaCl 0.15 M buffer. Glycans were eluated with a Tris 50 mM, NaCl 1 M buffer and dialysed on water.

Polymorphonuclear Neutrophil Isolation

Polymorphonuclear neutrophils (PMN) were isolated from blood by Ficoll Paque Plus gradient (GE Healthcare Bio-Sciences) followed by hypotonic saline lysis of erythrocytes in the granulocyte pellet. Purity of isolated PMN was 85-90% as determined by May-Grunwald Giemsa coloration. PMN were resuspended at a density of 10⁶ cells/ml in HBSS medium, without calcium or magnesium (Gibco), and activated by PMA (10 nM) for 3h at 37° C. Activated-PMN supernatants were collected, aliquoted and stored at −80° C.

Endocan Degradations

Preliminary studies have shown partial lost of endocan, as determined by ELISA, after defrosting or incubation at 37° C. This lost was abolished by addition of human serum albumin (HSA). Then, HSA was added to recombinant endocan at 10⁻²% in all degradation experiments.

Degradation of endocan was studied by incubating recombinant endocan with HSA and activated-PMN supernatant (10 to 20%), purified cathepsin G (Sigma), elastase (Sigma) or proteinase 3 (Sigma) at concentrations ranging from 1 ng/mL to 1 μg/mL for 2 h to 72 h.

Endocan degradation was evaluated by ELISA and western blot.

Action of Proteases Inhibitors

To check which neutrophil protease was responsive of the formation of which endocan degradation product, protease inhibition experiments were performed.

Protease inhibitors were pre-incubated with the proteases for 2 h at 37° C. before addition of endocan and HSA. The mixtures were then incubated for 24 h at 37° C. and endocan's degradation was determined by ELISA and western blot.

The proteases inhibitors used are: Complete cocktail without EDTA (Roche), 1 mM PMSF (Sigma), 1 mM Pefabloc SC (Invitrogen), 20 μM E-64 (Invitrogen), cathepsin G inhibitor I (Calbiochem) and elastase inhibitor III (Calbiochem).

ELISA

Endocan were quantified using a sandwich immunoassay with two monoclonal antibodies: MEP 14 (IgG2a/kappa) and MEC 15 (IgG1/kappa) which epitopes are respectively located at the C-terminal and N-Terminal extremity of endocan. These monoclonal antibodies can be produced by the hybridoma cell lines, respectively, I-1942 filled on Nov. 19, 1997, and I-2572 Oct. 17, 2000, before the Collection Nationale de Culture des Microorganismes de I'Institut Pasteur (CNCM), France (see also FR-2 775 691 and WO-02/39123).

MEP 14 mAb (2 μg/ml in carbonate buffer) was coated in 96-wells ELISA plates overnight at 4° C., and then washed and blocked with saturation buffer (PBS, BSA 0.1%, EDTA 5 mM, 0.1% Tween 20). After a washing step, endocan standards ranging from 10 ng/ml to 0.15 ng/ml or samples were added.

After an 1 h incubation at room temperature and washing step, the complexes were incubated with the MEC 15 mAb (0.1 μg/ml in saturation buffer), then washed and incubated with biotin anti-mouse IgG1 ( 1/20000, BD Biosciences Pharmingen).

Plates were washed again and streptavidin-HRP conjugate was added ( 1/10000, Zymed, San Francisco, USA) for 30 min at room temperature.

The complexes were finally washed and developed with TMB (Interchim) and the color reaction was stopped with H₂SO₄ 2N. The absorbance was determined at 450 nm on a spectrophotometer.

All washes and dilutions of standards, samples and antibodies, were done in saturation buffer. All antibodies incubations were done at room temperature for 1 h.

Western Blot

The size of endocan degradation products were determined by western blots. Samples were migrated on 15% reducing SDS-PAGE and blotted onto nitrocellulose membranes (Hybond ECL, Amersham Biosciences). After a blocking step, the membranes were incubated with monoclonal antibodies MEP 21 at 1 μg/ml, washed and then incubated with an anti-mouse Fc HRP-conjugated secondary antibody at 1/10000 (Sigma) followed by washes and developing by ECL detection kit (Pierce). Antibody incubations were done for 1 h at room temperature.

For the endocan p14 immunoblotting, the membrane was incubated after blocking step with MEP 21 HRP-conjugated monoclonal antibody ( 1/8000), washed and developed.

The monoclonal antibodies MEP 21 above-mentioned can be produced by the hybridoma cell line I-1944 filled on Nov. 19, 1997, before the Collection Nationale de Culture des Microorganismes de I'Institut Pasteur (CNCM), France (see also FR-2 775 691 and WO-02/39123).

Mass Spectrometry Analyses

To characterize the N-terminus of endocan p14, 2 μg of S137A/E and p14 derived from cathepsin G-treated endocan were deposited in a 15% acrylamide gel and stained by coomassie blue.

Protein bands corresponding to S137A/E and endocan p14 were excised from the gel, destained and digested by trypsin (ratio enzyme/substrate=1/100).

Obtained peptides were then extracted from the gel and analysed using a MALDI-TOF Voyager Elite mass spectrometry (Perspective Biosystem, Framingham, Mass., USA). The spectrometer operated in positive reflectron mode with an accelerating voltage of 20 kV and an extraction time of 200 nsec.

To characterize the C-terminus of p14, endocan (2 μg/mL) was incubated with cathepsin G (0.01 μg/mL) and HSA 10⁻²% (v:v) for 30 h at 37° C. to obtain a complete cleavage of endocan in p14.

After desalting and concentration, samples were diluted in matrice solution (1 μL of freshly dissolved 3-5-Dimethoxy-4-hydroxycinnamic acid at 10 mg/mL in 50% CH3CN, 0.05% TFA).

Next, samples were directed spotted onto the MALDI-TOF mass spectrometry target and the dried spots were analysed using a Voyager-DE-STR mass spectrometer (Applied Biosystems, Palo Alto, Calif., USA).

The spectrometer operated in linear mode with a positive accelerating voltage of 25 kV, a delayed extraction mode and an extraction time of 750 nsec.

The spectrometer was externally calibrated with the Calibration Mixture 3 of the Sequazime peptide Mass Standards Kit (Applied Biosystems) and the results spectra were analyzed with the Applied Biosystems Data Explorer software to determine the p14 mass.

Degradation of Endocan's Glycan

Purified endocan's glycan (5 μg) was incubated with various volumes of activated-PMN supernatant (0, 5, 50, 150 or 300 μl) for 15 h at 37° C. After drying in speed vac, samples were resuspended in glycerol 20% and deposited in a 30% acrylamide gel stained by blue alcian.

Heparin-Sepharose Binding Test

The activated-neutrophil supernatant was buffer exchanged in Tris 20 mM pH8 with PD-10 columns, following the recommendations of the manufacturer (GE Healthcare, Bio-sciences AB), and incubated in batch with Heparin-Sepharose (GE Healthcare, Bio-sciences AB) for 1 h 30 at 4° C.

After centrifugation, the flow through was collected and Heparin-Sepharose was washed twice with Tris 20 mM pH8 and then the proteins bound to the matrix were eluted with NaCl 1M buffer.

All the collected fractions (input, flow through, washings and elution) were then incubated with endocan for 24 h at 37°0 C., and samples were studied by western blot.

Competition Test Between Endocan or S137A/Endocan and Endocan's Glycan

Fifty ng of recombinant endocan or non glycanable endocan (S137A/endocan) were incubated with activated-PMN supernatant and increasing amounts of purified endocan's glycan (glycan:endocan ratio ranging from 0:1 to 50:1).

After 24 h at 37° C., samples were analysed by western blot with the anti-endocan mAb MEP 21.

Method for Producing Recombinant p14 Peptides

In order to obtain the three recombinant p14 peptides (i.e. recombinant p111, p115 and p116), the codons encoding for the amino acids K₁₁₂, F₁₁₆, and Q₁₁₇ in the cDNA of endocan isoform E16 were replaced by a stop codon, respectively by site-directed mutagenesis. Site-directed mutagenesis was performed with the Quick-Change site-directed mutagenesis kit, according to the manufacturer's recommendations (Stratagene).

Endocan isoform E16 refers to a variant of Endocan protein of SEQ ID N^{o}1 comprising the mutation F116A (the phenylalanine at 116 is replaced by alanine). The resulting nucleic acids encoding for p111, p115 and p116 correspond to the nucleic acid sequences SEQ ID N^{o}3, SEQ ID N^{o}4 and SEQ ID N^{o}5, respectively.

Each resulting nucleic acid was inserted into pcDNA3.1 DHFR vector (BL16-DHFR2-T7_D04_{—}026.ab1 ABIX Testing).

TABLE 1
Differences between E16 cDNA and the resulting
nucleic acids encoding for p111, p115, or p116
peptides. The nucleotides written in bold
correspond to the nucleotides mutated by site-
directed mutagenesis to generate the three
recombinant p14 peptides. For obtaining p116,
it should be noticed that the codon GCC coding
for A116 was replaced by the codon TTC
encoding for phenylalanine.

| | | Nucleic acid encoding p14 |
|---|---|---|
| | | peptides generated by site-directed |
| | E16 cDNA | directed mutagenesis |
| p111 | . . . NNN CTG AAA NNN . . . | . . . NNN CTG TAA NNN . . . |
| | . . . AA L₁₁₁ K₁₁₂ AA . . . | . . . AA L₁₁₁ Stop |
| p115 | . . . NNN TTC GCC NNN . . . | . . . NNN TTC TAA NNN . . . |
| | . . . AA F₁₁₅ A₁₁₆ AA . . . | . . . AA F₁₁₅ Stop |
| p116 | . . . NNN GCC CAA NNN . . . | . . . NNN TTC TAA NNN . . . |
| | . . . AA A₁₁₆ Q₁₁₇ AA . . . | . . . AA F₁₁₆ Stop |

Competent TOP10F′ E. coli (Invitrogen) were transformed with the pcDNA3.1 p111, p115 or p116 vectors to amplify these plasmids. The plasmids were purified in a following step according to conventional methods. Each of these three plasmids was used to transfect CHO DG44 cells in the presence of Lipofectamine 2000 (Invitrogen). The CHO DG44 cells were routinely cultured in alpha-MEM supplemented with 10% FCS, HT-Supplement and 2 mM L-glutamine. Stably transfected cells were selected by the culture medium containing alpha-MEM supplemented with 10% desalted FCS and 2 mM L-glutamine. The recombinant p111, p115 and p116 peptides were then isolated following conventional purification methods (such as methods comprising immunoaffinity and/or ion exchange chromatography) from the stably transfected cell culture.

Method for Producing Polyclonal Antibodies Directed to p14 Peptides

In order to obtain antibodies which are specific to p14 peptides and which do not detectably bind to endocan, Balb/c mice and Lewis rats were immunized with two types of immunogenic compounds resulting from the coupling of KLH with a C-terminal fragment of the p116 peptide (Genepep, Montpellier, France).

The first immunogenic compound resulted from the coupling of KLH with the “peptide 1” having the amino acid sequence of SEQ ID N^{o}6 (which corresponds to the amino acid sequence 104-116 of SEQ N^{o}1).

The second immunogenic compound resulted from the coupling of KLH with the “peptide 2” having the amino acid sequence of SEQ ID N^{o}7 (which corresponds to the amino acid sequence 108-116 of SEQ N^{o}1).

Peptide 1 and Peptide 2 were obtained by a conventional solid-phase synthesis (Genepep, Montpellier, France)

Conjugates with BSA were also obtained for each peptide.

For each KLH-peptide, 2 Balb/c mice and 2 Lewis rats were immunized with a dose of 10 μg and 50 μg, respectively. The regimen was a first injection in complete Freud adjuvant, and then, one injection every 3 weeks with incomplete Freud adjuvant. Two weeks after the second injection, blood samples were checked for an antibody response to the immunogenic compounds.

For this purpose, BSA-peptide 1 conjugate or BSA-peptide 2 conjugate (2 μg/mL) was adsorbed on 96-well microplates overnight at 4° C. The wells were then saturated by incubation of ELISA buffer (PBS, BSA 0.1%, EDTA 5 mM, 0.1% Tween 20) for 1 hour at room temperature (RT). The pre-immune and immune serums were diluted in ELISA buffer and then incubated for 1 h at RT. After washings, the bound antibodies were revealed by either an HRP-labeled anti-mouse IgG antibody ( 1/5000) or an HRP-labeled anti-rat IgG2a monoclonal antibody ( 1/3000). After washings, the plates were revealed by TMB and the reaction was stopped with 2N H₂SO₄, and read at 450 nm.

The obtained results are shown in FIGS. 7A and 7B; the values are done in optical density unit.

Evaluation of the Specificity of Polyclonal Antibodies (Competitive Immunoassay)

In order to confirm that the immune serums recognize specifically p14 peptides but not endocan wild type, competitive immunoassay was performed.

96 well ELISA microplates were coated with BSA-peptide 1 conjugate (2 μg/ml). The wells were then saturated by incubation of ELISA buffer (PBS, BSA 0.1%, EDTA 5 mM, 0.1% Tween 20) for 1 hour at room temperature (RT). The preimmune and immune serums from 2 rats were diluted from 1:1000 to 1:8000 (V:V) with either recombinant endocan (200 ng/mL in ELISA buffer), crude CHO-DG44 cell supernatant containing recombinant p111, p115 or p116 (transient transfection), or crude control cell supernatant (HEK-endocan cells or fresh culture medium).

Then, the complexes were added to the wells of ELISA plates and incubated one hour at RT. After washings, the bound antibodies are revealed by an HRP-labeled anti-rat IgG2a monoclonal antibody ( 1/3000). After washings, the plates are revealed by TMB and the reaction stopped with 2N H2SO4, and read at 450 nm. The values are done in optical density unit (n=2).

After washings, the bound antibodies are revealed by an HRP-labeled anti-rat IgG2a monoclonal antibody ( 1/3000). After washings, the plates are revealed by TMB and the reaction stopped with 2N H₂SO₄, and read at 450 nm. The values are done in optical density unit (n=2).

Method for Producing Monoclonal Antibodies Directed to p14 Peptides

Monoclonal antibodies may be generated by immunization of lewis rats or Balb/c mice with purified KLH-peptide 1 conjugate or purified KLH-peptide 2 conjugate as previously described.

Blast cells from inguinal draining lymph nods may be fused with Sp2/0 myeloma cells.

Clonal hybridoma cells may be screened to recognize peptide 1 or peptide 2 as illustrated above. Then hybridoma cells which react with p14 peptides and has low affinity or no affinity for endocan of SEQ ID N^{o}1 may be selected. Hybridoma cells may be cultured in RPMI 1640 supplemented with 10% FCS, 5 mM HEPES, HT-Supplement or a serum-free Hybridoma-SFM medium (Invitrogen). Specific anti-p14 monoclonal antibodies may be purified from the supernatant of said hybridoma cells according to conventional purification methods.

Results

Specific Proteolysis of Endocan by Neutrophil Elastase and Cathepsin G

It was first studied the degradation of endocan by PMN supernatants, indicating that PMN supernatants decrease the level of endocan, determined by ELISA.

However, supernatants from PMN activated for 3 hours with PMA showed 10 times more degradation activity than those from resting PMN.

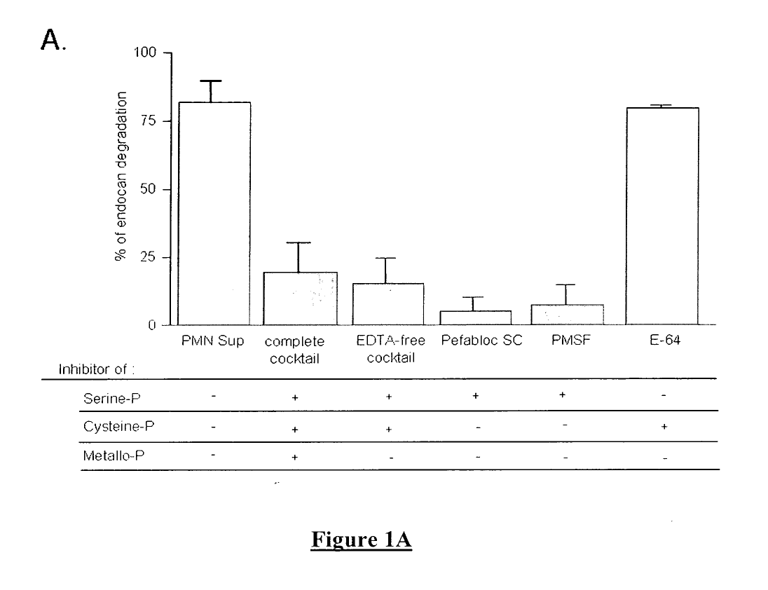

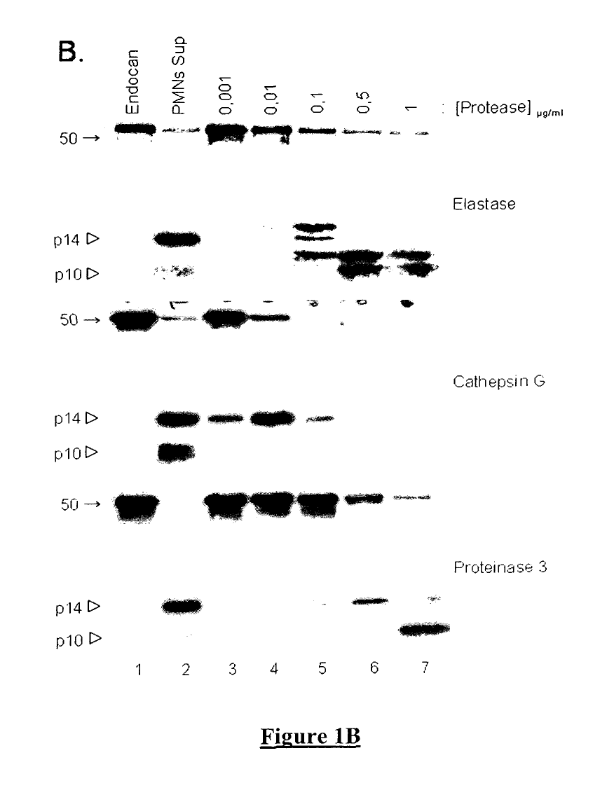

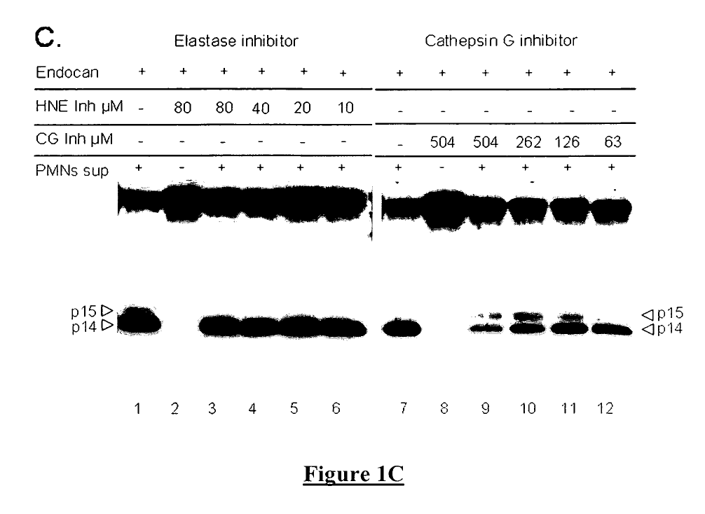

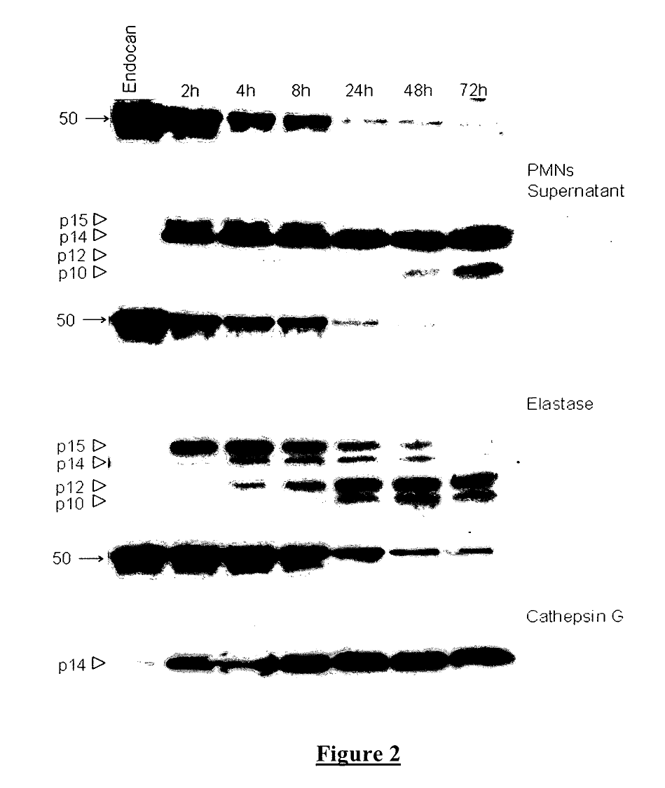

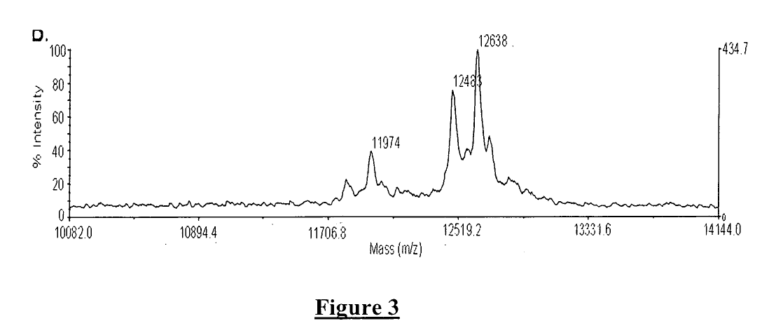

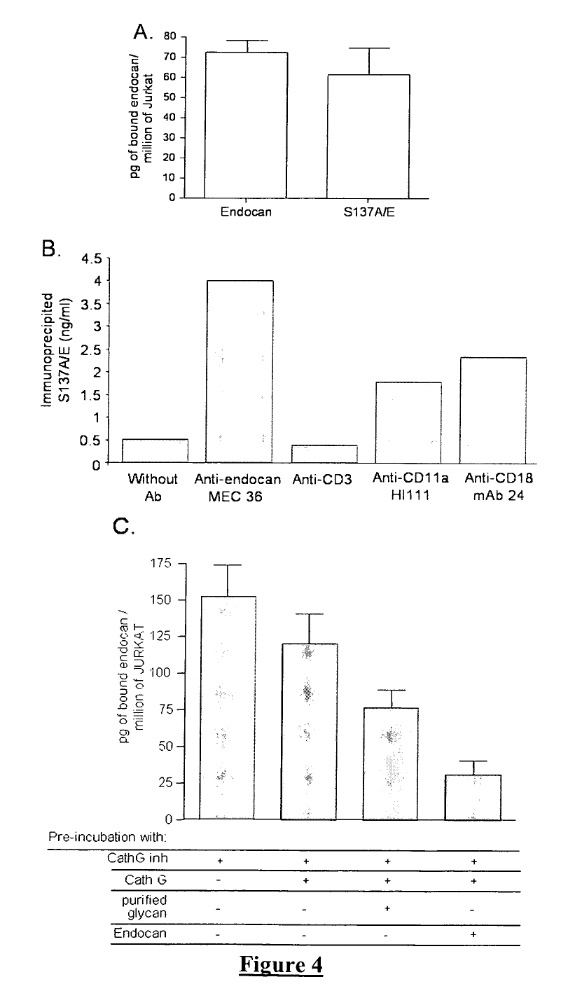

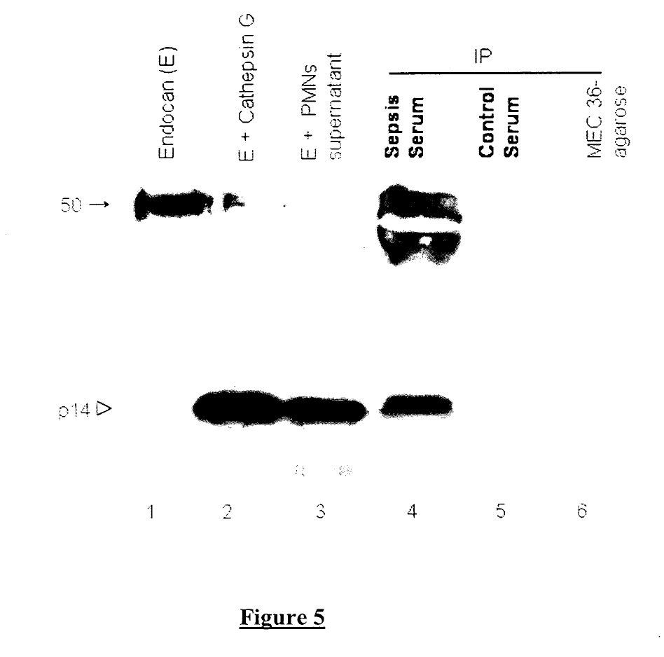

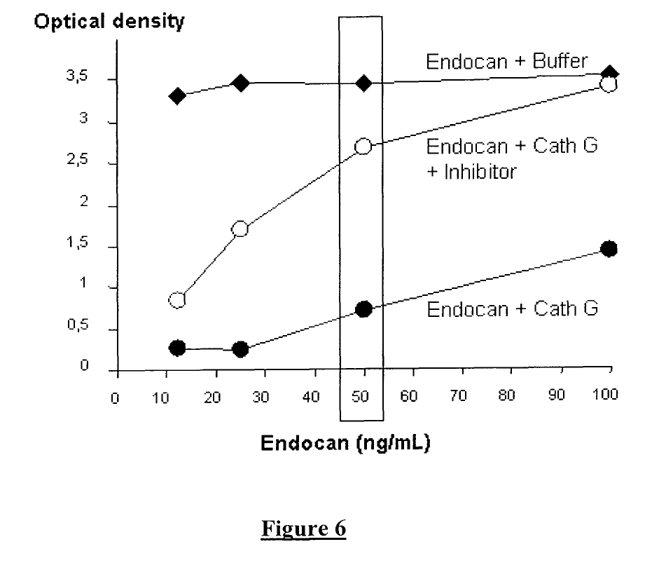

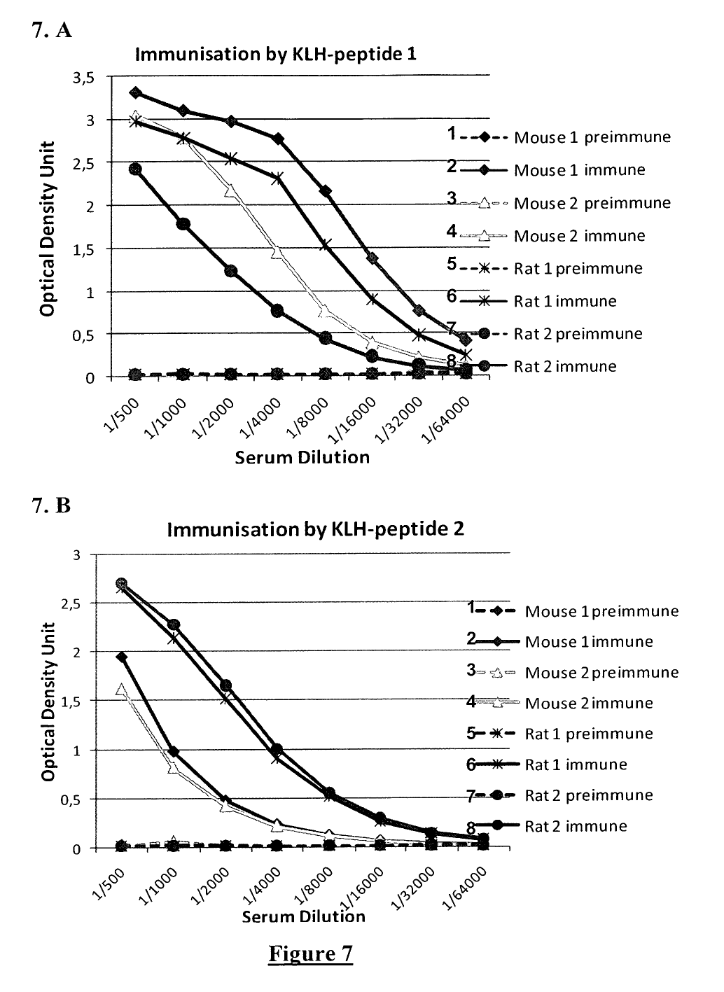

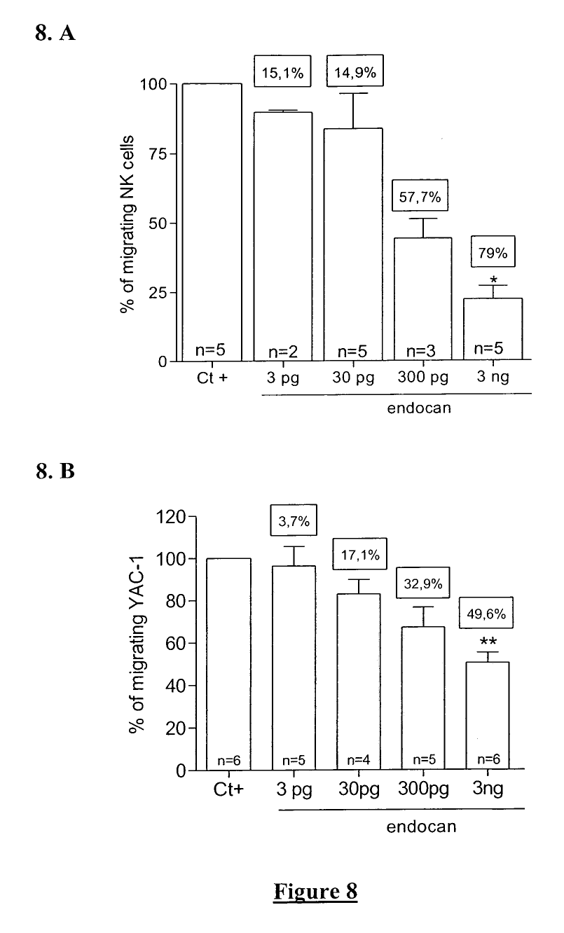

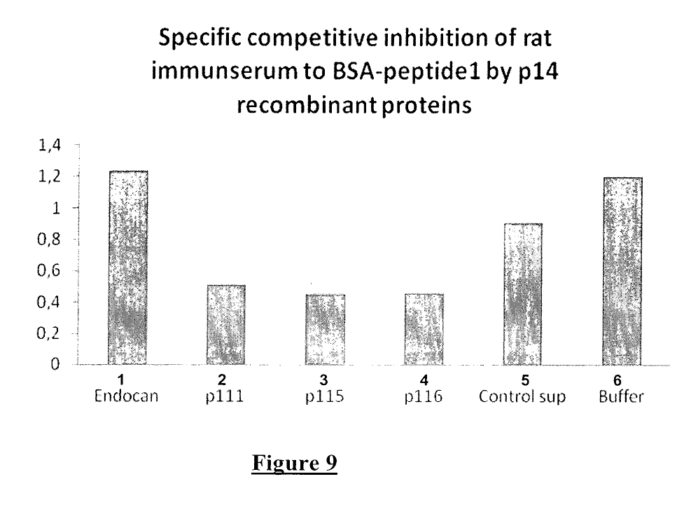

In conditions including 2 μg/mL endocan and 10% activated neutrophil supernatants at 1×10⁶ cells/mL, 24 h incubation at 37° C. is sufficient to reduce up to 90% the level of endocan (FIG. 1A). Addition of a complete protease inhibitor cocktail prevented the lost of endocan induced by PMN supernatants (FIG. 1A).

In order to determine which enzyme family is involved, inhibitors of serine proteases (Complete cocktail, EDTA free-cocktail, PMSF, Pefabloc SC), cysteine proteases (EDTA free-cocktail, E-64) and metalloproteases (Complete cocktail, EDTA) were used. The FIG. 1A shows that only the inhibitors of serine proteases are able to inhibit proteolysis of endocan by the activated-neutrophil supernatants.

It was next examined the endocan degradation products by western blot. 24 h incubation with PMN supernatants resulted in the loss of native endocan at 50 kDa, and the appearance of 2 bands of 14 kDa and 10 kDa (FIG. 1B, lane 2). These results suggested that PMN activation induces secretion of serine proteases which in turn degrade endocan.

Polymorphonuclear neutrophils secrete three serine proteases: elastase, cathepsin G and proteinase 3. Therefore, we tested the activity for each of these purified proteases towards endocan.

Incubation of endocan with elastase for 24 h induced 4 bands of 15, 14, 12 and 10 kDa. Increasing concentrations of elastase resulted in a decrease of 15 and 14 kDa bands and an increase of 12 and 10 kDa. The minimal concentration of elastase required for near complete degradation of endocan was 0.1 μg/mL (FIG. 1B).

Incubation of endocan with cathepsin G induced only one band of 14 kDa. The minimal concentration of cathepsin G required to cleave completely endocan in 24 h was 0.01 μg/mL. Concentrations up to 0.1 μg/mL resulted in the loss of the 14 kDa band (FIG. 1B).

Incubation of endocan with proteinase 3 resulted in the loss of the 50 kDa band only with high concentration of proteinase 3 up to 1 μg/mL (FIG. 1B). Such a high enzyme concentration suggested that endocan is relatively resistant to proteinase 3 proteolysis. Then, our work focused on elastase and cathepsin G.

To confirm that elastase and cathepsin G from PMN supernatants are exclusively involved in the degradation of endocan, specific inhibitors were used. Addition of elastase inhibitor III to PMN supernatants led to the absence of endocan p15, but did not prevent the formation of endocan p14 (FIG. 1C, lanes 3-6).

On the other hand, addition of cathepsin G inhibitor I did not prevent the formation of endocan p15, but reduced, dose-dependently, the formation of endocan p14 (FIG. 1C, lanes 9-12).

Moreover, 1 mM cathepsin G inhibitor I completely inhibited endocan degradation as determined by ELISA.

Taken together, these results suggested that PMN secrete proteases, among which elastase and cathepsin G are exclusively involved in the catabolic process of endocan, resulting in several degradation products from 10 to 15 kDa.

Cathepsin G Generates a Major and Sustained Degradation Product p14

Thanks to kinetics of endocan degradation by the activated PMN supernatants, elastase, and cathepsin G, we determined the fate and stability of each product degradation over time.

In the presence of PMN supernatants, endocan is cleaved in 15 and 14 kDa fragments from 2 hours of incubation.

While p15 disappeared over 8 h incubation, new fragments of 12 and 10 kD appeared from this time and increased until 72 h incubation.

Endocan p14 remained stable and represented the major product till the 72th hour of contact with PMN supernatant.

Kinetics of endocan degradation by elastase showed that endocan is cleaved in a sequential manner in peptides of 15 and then 12 and 10 kDa. However, the kinetics of degradation by cathepsin G showed that this protease cleaved endocan in a single and stable fragment of 14 kDa, from 2 to 72 hours incubation.

This result confirmed that the sum of the degradation profiles of endocan by elastase and by cathepsin G, matched well with that obtained by PMN supernatants. One surprising observation was that cathepsin G generates the major endocan degradation product p14. Another surprising point was that this endocan p14, even in the presence of elastase, remained present all over the time course of the kinetics.

Characterization of the Endocan p14.

To analyse the N-terminus of endocan p14, trypsic fingerprinting were compared between endocan p14 produced by PMN supernatants and non glycanable endocan S137A/E.

The results indicated that the N-terminal aminoacids from 1 to 105 of endocan p14 are identical to that from S137A/E. The aminoacids 145-165 are absent, suggesting that the cleavage site is located within the aminoacids 106-144 (See Table 1).

The determination of the C-terminus of endocan p14, derived from cathepsin G-treated endocan, was deduced from the mass obtained by MALDI-TOF spectrometry (FIG. 3). The results showed 3 putatives polypeptides of 11974, 12483, 12638 Daltons. These polypeptides corresponded to the first 111, 115 and 116 amino acids, respectively.

To conclude, these results demonstrate that the N-terminus of endocan is not cleaved by PMN supernatant.

These results also show that Cathepsin G is able to cleave endocan in three cleavage sites, i.e. between the amino acids L₁₁₁/K₁₁₂, F₁₁₅/F₁₁₆ and F₁₁₆/Q₁₁₇ of endocan to give the three p14 proteins : p111, p115 and p116 (Table 1).

TABLE 2
Characterization of the three p14 fragments
generated by Cathepsin G,

| | Mass of | | | Endocan |
|---|---|---|---|---|
| Peptide | the p14 | Number of | Protein | peptide bond |
| name | peptide (Da) | amino acids | sequence | cleaved by CG |
| p111 | 11974 | 111 | W₁ to L₁₁₁ | L₁₁₁/K₁₁₂ |
| p115 | 12483 | 115 | W₁ to F₁₁₅ | F₁₁₅/F₁₁₆ |
| p116 | 12638 | 116 | W₁ to F₁₁₆ | F₁₁₆ /Q₁₁₇ |

W = Tryptophan;
L = Leucine;
F = Phenylalanine;
K = Lysine;
Q = Glutamine

Immunization of Mammals with Carrier Protein—C-Terminal Fragment of p14 Induces the Production of Antibodies Directed to Said Fragment

As illustrated in FIGS. 7A and 7B, the immunization of mice and rats with KLH coupled with peptide 1 of SEQ ID N^{o}6 or with peptide 2 of SEQ ID N^{o}7 induces a significant production of polyclonal antibodies directed to peptide 1 and peptide 2, respectively. Pre-incubation of the immune serums with synthetic peptide 1 or peptide 2 reduced the binding of antibodies to coated immunogenic compounds. It was also shown that the polyclonal antibodies of the immune serum do not bind to absorbed BSA. Taken together, these results underline the high specificity of the produced polyclonal antibodies.

Polyclonal Antibodies from Immune Serum are Specific to p14 Peptides and Does Not Bind to Endocan

The 2 immune serums bound to BSA-peptide 1 (FIG. 9, ELISA buffer). The presence of control supernatant or endocan wild type did not modify the antibody binding to the BSA-peptide 1 (FIG. 9, Endocan, control sup.). By contrast, incubation with cell supernatants containing p111, p115 or p116 significantly reduced the antibody binding to the BSA-peptide 1 (FIG. 4, p111, p115, p116). We concluded that the antibodies raised against the peptide 1 are able to distinguish p14 from endocan wild type (SEQ ID N^{o}1).

### Example 2

### Detection of Endocan and Endocan p14 in Human Serum

A. Materials and Methods

Native endocan is detected by ELISA (Scherpereel A et al, Crit Care Med. 2006;34:532-537). The presence of p14 in human serum of patients with septic shock was determined by immunoprecipitation combined with western blot.

The volume of sera or plasma from two patients with sepsis were pooled to give about 100 ng endocan (the sera provided from the collection previously published in Scherpereel A et al, Crit Care Med. 2006;34:532-537). The sera were cleared by centrifugation at 3,000 g for 15 min, followed by filtration at 0.45 μm, and then diluted 1:3 (v/v) in PBS containing anti-protease mix (Roche). For immunoprecipitation, the anti-endocan mAb MEC 36 was coupled to an agarose support matrix, using the recommendations of the Affi-Gel Hz immunoaffinity kit (Bio-Rad). Hundred μL MEC36-agarose beads were added to the sera overnight at 4° C. under constant agitation. Agarose beads were collected by centrifugation, washed 3 times with PBS/0.5% NP 40/anti-proteases cocktail and then 3 times with PBS containing anti-proteases. After centrifugation, the beads were resuspended in 50 μl of SDS-PAGE sample buffer, DTT was added and samples were studied by western blot with the MEP 21 HRP-conjugated monoclonal antibody.

A sandwich ELISA assay can be performed as an alternative method for detecting and quantifying p14 peptides in human serum. In this assay the primary antibody should be a specific anti-p14 peptide antibody which does not bind endocan. Said specific anti-p14 peptide antibody can be obtained according to Example 2 (see paragraph “Method for producing monoclonal antibodies directed to p14 peptides” in Material and Methods). The secondary antibody may be directed against the N-terminus of endocan. The detection and quantification of sandwich complex may be performed thanks to a HRP-coupled anti-Fc antibody.

B. Results

Both Native Endocan and Endocan p14 are Present in Serum from Septic Shock Patients

Serum endocan levels are increased in septic shock patients, and PMN are well known to play a critical role in the features of this disease. Then, it was tested if endocan p14 could be detected in serum from septic shock patients.

To explore this, blood samples from several patients with septic shock were pooled to give a total amount of 100 ng native endocan, determined by ELISA.

Immunoprecipitation of endocan from this pooled serum revealed a large band around 50 kDa, which corresponded to native endocan (FIG. 5, lane 4).

In addition, it revealed a second band (FIG. 5, lane 4) with the same molecular weight to the p14 derived from cathepsin G-treated endocan (FIG. 5, lane 2), or the activated PMN supernatants (FIG. 5, lane 3).

In contrast, no such a degradation fragment was immunoprecipitated from serum of healthy subjects (FIG. 5, lane 5).

This result demonstrates that during the septic shock syndrome, 2 forms of endocan are detected in serum: the native endocan and its major degradation product p14, which represents a specific product of cathepsin G derived from PMN.

C. Clinical Research Protocol

Main Objective:

To estimate the blood levels of endocan and p14 peptides in order to predict the clinical severity and the prognosis for patients admitted in intensive care unit for acute sepsis or septic shock.

This clinical research trial will comprise the kinetics study of the levels of circulating native endocan and those of p14 peptides relating to the clinical status of the patients on treatment (estimated by acute physiology scores such as SAPS-II) and their fate (i.e. the evolution to ARDS or to Multiple organ dysfunction syndrome: survival or death on day 10^{th} or on day 28^{th} of hospitalization).

For comparative purpose, the same parameters will be determined for patients who suffer from “medically” inflammatory state i.e. for a group of patients who will be tested before and after abdominal surgery.

Protocol

The clinical research trial will be a longitudinal and prospective study which will comprise comparisons between unpaired groups of patients admitted in intensive care and suffering from acute sepsis (n=100) relating to a control group (voluntary healthy patients), and between pathological groups. Blood collection will be performed at the admission of the patient in intensive care (T0) and at 12 hours, 24 h, 48 h, 5 days, 7 days, 10 days, 14 days, 20 days and 28 days of hospitalization (i.e. 10 samples for each patient). The blood level of endocan and p14 peptides as well as the blood level of other inflammatory soluble biomarkers will be measured in order to evaluate the status of the patient at the moment of the blood collection and in order to compare the said blood levels with those described in the prior art.

The blood level of p14 peptides will be determined by immunoassay, preferably by sandwich ELISA based on the used of a specific anti-p14 antibody as primary antibody.

The measured blood levels for the different biomarkers will be compared with the clinical data and the degree of sepsis severity for each patient. The main objectives of the study will be

(i) to evaluate the specific variations of endocan and p14 levels according to human septic pathology and

(ii) to illustrate the utilization of the blood level variations of endocan and p14 for estimating the prognosis of patients on treatment and

(iii) to compare the levels of endocan and p14 with those of other inflammatory markers or those of cellular activation markers.

Another control group will be also included in this study. This group will consist of surgical patients (n=20). The protocol of blood collection and assay will be the same that of group of septic patients. The blood collection at T0 will be performed just before the surgical operation.

Such methodology will enable to compare septic patients with patients having non-infectious vascular damage and inflammatory acute state.

### Example 3

A. Materials and Methods

Binding Test on Jurkat Cells

It has been previously demonstrated that endocan binds to Jurkat cell surface through interaction with CD11a/CD18 integrin. This test is used to compare the binding of endocan and its non glycanable mutant (S137A/E) to leukocytes, 10×10⁶ Jurkat were incubated with 2 ml of supernatant culture cells containing 300 ng/ml of endocan or S137A/E (RPMI 1640 medium supplemented with 10% FCS, 2 mM L-Glutamine and Ticarpen) for 1 h at 4° C. Jurkat were centrifuged at 1600 rpm for 5 min at 4° C., washed 3 times with ice-cold RPMI medium and lysed at 4° C. for 30 min with 500 μl of lysis buffer containinig PBS, 0.5% Nonidet P-40 (Roche) and a cocktail of proteases inhibitors with EDTA (Roche). After centrifugation at 400 g for 10 min at 420 C., bound endocan and S137A/E were quantified by ELISA.

Immunoprecipitation of Jurkat lysates containing endocan or non glycanated endocan S137A/E was performed as previously described in Bechard D et al (J Immunol. 2001;167:3099-3106) using anti-CD11a mabs HI111 (BD Biosciences), and mab24 (a generous gift of Dr Nancy Hogg from Leukocyte Adhesion Laboratory, Cancer Research UK, London Research Institute, London, UK).

To evaluate if endocan p14 could influence endocan binding to Jurkat cells, the cells were first incubated with p14, endocan's glycan or the buffer supplemented with MnCl₂ and CaCl₂ at 1 mM each for 1 h at 4° C. Then cell supernatant- containing endocan were added, and Jurkat cells were next washed three times with PBS and lysed. For these experiments p14 derived from endocan degradation by cathepsin G. To make sure that protease activity did not cleave endocan from the supernatant culture, we added the specific cathepsin G inhibitor I (Calbiochem).

Assay to Evaluate the Leucocyte-Transendothelial Cell Migration in the Presence of Endocan

In the model of leukocyte—transendothelial cell migration, human primary endothelial cells (HUVECs) were cultured in Transwell filters. Two types of leukocytes were used in this migration assay:

(a) human. NK cells which are a primary leukocyte population purified from blood, and

(b) the murine lymphoid T cell line YAC-1.

The primary human endothelial cells HUVECs were cultured on Transwell for 3 days in order to obtain a continuous cell monolayer. The HUVECs were stimulated by TNF for 3 hours. Then the chemokine SDF1alpha is added to the well, and the leukocytes (5×10⁵ cells by Transwell) were added to the Transwell. Before their adding to the transwell, the leucocytes were incubated 3 h at 37° C. with:
(a) monoclonal anti-human LFA-1 clone HI111 (anti-LFA-1); monoclonal anti-mouse LFA-1 clone H155-141 (anti-mu-LFA-1) or) or a monoclonal isotype control (Ct iso); or
(b) with increasing amount of recombinant endocan of SEQ ID N^{o}1 (3 pg/ml, 30 pg/ml, 300 pg/ml, and 3 ng/ml).

The positive control (Ct+) was performed in the presence of the buffer alone (without endocan and antibodies).

After 3 h incubation at 37° C. the number of leukocytes that had migrated to the well were counted. The percentage of leukocyte migration was obtained by the ratio of the number migrated leukocytes in the test sample (i.e. in the presence of endocan or antibodies) to the number of migrated leukocytes in the positive control (Ct+).

The encadred values are mean inhibition calculated as follows: Inhibition=1−[(Sample −random migration)/(Ct+−random migration)]*100.

Assay to Screen Inhibitors of Cathepsin G—Endocan Interactions

Hundred μL of anti-endocan C-ter antibody MEP14 in carbonate buffer was coated at 5 μg/mL overnight at 4° C. in 96-well microtiter plates. The plates were washed with PBS and saturated with PBS containing 0.1% BSA and 5 mM EDTA (ELISA buffer). Recombinant human endocan was incubated one hour at room temperature under constant agitation at various concentrations ranging from 12.5 to 100 ng/mL. After 3 washings, 100 μL of 0.5 μg/mL to purified cathepsin G (Sigma) or Cathepsin G+30 μM cathepsin G inhibitor I (Calbiochem) in ELISA buffer was added, and then incubated for 2 hours at 37° C. The plates were then washed 3 times with ELISA buffer containing 0.1% Tween 20 (saturation buffer). Residual bound endocan was revealed by incubation of 0.1 μg/mL anti endocan-N-ter antibody MEC15 for 1 hour, followed by streptavidin-HRP conjugate ( 1/10000, Zymed, San Francisco, USA) for 30 min, and developed with TMB (Interchim). The absorbance was determined at 450 nm on a spectrophotometer. All washes and dilutions of standards, samples and antibodies, were done in saturation buffer.

B. Results

Endocan p14 Inhibits Binding of Endocan to the Jurkat Cells

Endocan p14 derived from the degradation of endocan by cathepsin G is major, stable and accounts for nearly ¾ of the endocan protein core. Therefore, it was studied if this p14, as endocan, could bind to Jurkat cells.

Previous studies have shown that endocan binds to Jurkat cells via the LFA-1 integrin. However, whether endocan binds to LFA-1 through its glycan or its protein core is unknown. Our results showed that endocan and S137A/E bound similarly to the surface of Jurkat cells (FIG. 4A).

Immunoprecipitation of Jurkat cell lysates containing endocan or S137A/E with anti-CD11a mab HI111 co-precipitated S137A/E as well as endocan (FIG. 4B).

In addition, similar results were obtained with another anti-CD11a reporter epitope recognized by mab24.

Thus, our results indicate that endocan binds through its protein core to LFA-1 rising the hypothesis that p14, which corresponds to ⅔ native endocan protein core, could also bind to LFA-1 (FIG. 4B).

We speculated that p14 could inhibit binding of native endocan to Jurkat cells. Then, p14 was generated by treatment of recombinant endocan with cathepsin G followed by its inhibition with specific cathepsin G inhibitor.

Indeed, pre-incubation of Jurkat cells decreased the binding of endocan to Jurkat cells, compared to the binding of endocan obtained with the buffer control and cathepsin G control (FIG. 4C).

However, with the endocan's glycan, a slight inhibition of endocan binding was observed.

These results suggested that p14 inhibits the binding of endocan to Jurkat cells through a competition binding to the endocan's receptor, the leukocyte integrin LFA-1.

Endocan Acts as Endogenous Inhibitor of LFA-1 Dependent Leukocyte—Transendothelial Migration Cascade

In the presence of anti-LFA-1 antibodies, the transendothelial migration of NK and YAC-1 cells was reduced by 82% and 56% respectively. Such inhibition was not observed in the presence of monoclonal isotype control (Ct iso). This result clearly shows that the migration of leucocytes is dependent on LFA-1.

The presence of recombinant endocan induced a dose-dependent inhibition of transendothelial cell migration of NK (see FIGS. 8A and 8B). In the presence of 3 ng of endocan, the percentage of cell migration inhibition reached 79% and 49.6% for NK cells and YAC-1, respectively which is similar to the maximal percentages of cell migration inhibition observed in the presence of anti-LFA-1 antibodies.

In addition to the previous works, the present results support the statement that endocan, spontaneously released by endothelial cells, binds to its receptor, namely the leukocyte integrin LFA-1, reduces the LFA-1/ICAM-1 interaction and thus inhibits the LFA-1-dependent transendothelial cell migration of leukocytes.

The fact that endocan acts as an endogenous inhibitor of LFA-1-dependent leukocyte migration clearly shows its anti-inflammatory activity.

Assay to Screen Inhibitors of Cathepsin G—Endocan Interactions

As illustrated in the above results, endocan certainly plays an anti-inflammatory activity by inhibiting the LFA-1-dependent transendothelial cell migration of leukocytes. Consequently, a molecule which can inhibit the degradation of endocan is likely to act as an anti-inflammatory compound in vivo. Such compound can be very useful for developing a treatment for acute and inflammatory diseases associated with activated polymorphonuclear neutrophil leukocytes.

In this context, an assay for screening anti-inflammatory candidate substances which inhibit the interaction of endocan with cathepsin G was developed (see “material and method” part of Example 3).

The following results were obtained:
Two hours incubation of cathepsin G at 37° C. appear ed sufficient to reduce the detection of bound endocan from 70% to 100%. Addition of cathepsin G inhibitor prevented partially the loss of endocan signal.
The best difference was observed with endocan at 50 ng/mL shown in the box of FIG. 6.

TABLE 3
Characterization of endocan p14 peptide

| | | Relative intensity | |
|---|---|---|---|
| Amino acid | peptide | | Endocan |
| Position | Mass | S137A/E | p14 |
| 1-19 | 2356.9 | 5% | 5% |
| 26-36 | 1411.58 | 2% | Absent |
| 27-36 | 1255.48 | 65% | 40% |
| 37-42 | 633.31 | 9% | 2% |
| 37-48 | 1399.62 | 5% | 5% |
| 43-48 | 785.32 | 60% | 25% |
| 58-63 | 659.32 | 20% | 5% |
| 64-81 | 2070.85 | 3% | 2% |
| 82-93 | 1478.54 | 60% | 25% |
| 94-105 | 1499.56 | 55% | 20% |
| 113-122 | 1263.64 | 5% | Absent |
| 113-126 | 1707.84 | <1% | <1% |
| 145-159 | 1615.81 | 12% | Absent |
| 150-159 | 1031.49 | 5% | Absent |
| 160-165 | 813.47 | 35% | Absent |
| 161-165 | 685.34 | 100% | Absent |

In table 1 above:
“Amino acid position” means the expected peptide;
“Relative Intensity” means peak intensity with (i) 100%=685 mass for S137A/3 and (ii) 100%=842mass for endocan p14.

For obtaining the data reported in Table 1, S137A/E and endocan p14 (derived from cathepsin G-treated endocan) were deposited in an acrylamide gel, stained by coomassie blue. The bands corresponding to each protein were excised from the gel, destained, digested by is trypsin and analysed by MALDI-TOF mass spectrometry.

TABLE 4
Sequences included in the sequence listing

| SEQ ID NO: | Sequences |
|---|---|
| 1 | Human active Endocan (1-165) |
| 2 | cDNA encoding for human full-length endocan |
| | (1-184) |
| 3 | Nucleic acid encoding for p111 resulting from |
| | site-mutagenesis of E16 cDNA |
| 4 | Nucleic acid encoding for p115 resulting from |
| | site-mutagenesis of E16 cDNA |
| 5 | Nucleic acid encoding for p116 resulting from |
| | site-mutagenesis of E16 cDNA |
| 6 | Peptide 1, a C-ter fragment of p116 |
| 7 | Peptide 2, a C-ter fragment of p116 |

1. An enzyme proteolysis-resistant peptide that binds to antibodies directed against the amino acid region 1-116 of the endocan's polypeptide sequence of SEQ ID N^{o} 1, which peptide possesses an apparent molecular weight of 14 kDa.

2. The peptide according to claim 1, which is cathepsin G-resistant and binds to MEP21 antibodies that are produced by the hybridoma cell line deposited on 19 Nov. 1997 at the CNCM under the accession number I-1944.

3. The peptide according to claim 1, which is selected from the group of peptides consisting of
(i) a peptide having a MALDI-TOF mass of 11974 Daltons,
(ii) a peptide having a MALDI-TOF mass of 12483 Daltons, and
(iii) a peptide having a MALDI-TOF mass of 12638 Daltons.

4. The peptide according to claim 1, which is selected from the group of peptides consisting of
(i) a peptide having the amino acid sequence 1-111 of the human endocan sequence of SEQ ID N^{o} 1,
(ii) a peptide having the amino acid sequence 1-115 of the human endocan sequence of SEQ ID N^{o} 1, and
(iii) a peptide having the amino acid sequence 1-116 of the human endocan sequence of SEQ ID N^{o} 1.

5. A recombinant peptide selected from the group consisting of
(i) a recombinant peptide having at least 90% amino acid identity with the amino acid sequence 1-111 of the human endocan sequence of SEQ ID N^{o} 1,
(ii) a recombinant peptide having at least 90% amino acid identity with the amino acid sequence 1-115 of the human endocan sequence of SEQ ID N^{o} 1, and
(iii) a recombinant peptide having at least 90% amino acid identity with the amino acid sequence 1-116 of the human endocan sequence of SEQ ID N^{o} 1

6. A monoclonal antibody to one or more peptides as defined in claim 1, the said monoclonal antibody does not detectably bind to human endocan having the amino acid sequence of SEQ ID N^{o}1.

7. A method for determining the occurrence of an inflammatory state in a subject, comprising the steps of:
a) providing a sample previously collected from the said subject,
b) measuring the amount value of at least one peptide according to claim 1, in the said sample,
c) determining the inflammatory state of the said subject from the peptide amount value measured at step b).

8. The method according to claim 7,
wherein step b) further comprises measuring the amount value of human endocan of SEQ ID N^{o} 1 in the said sample, and
wherein, in step c), consists of determining the inflammatory state of the said subject from the ratio of the amount values of (i) the at least one peptide measured at step b), to (ii) the human endocan of SEQ ID N^{o} 1 measured at step b).

9. The method according to claim 7, wherein the said inflammatory state is selected from the group consisting of a chronic inflammatory state and an acute inflammatory state.

10. The method according to claim 7, wherein the said inflammatory state is selected from the group consisting of sepsis, acute sepsis and septic shock.

11. The method according to claim 7, wherein the inflammatory state is selected from the group consisting of acute lung injury (ALI) or acute respiratory distress syndrome (ARDS).

12. The method according to claim 7, wherein step b) is performed by determining the intensity value of the signal that is generated by the said peptide biomarker when the said sample is subjected to a mass spectrum analysis or an immunoassay analysis.

13. A method for monitoring the treatment efficiency of a patient affected with an inflammatory state, comprising a step of performing the method according to claim 7 with one or more samples that have been collected from the said patient at one or more instants.

14. A method for the in vivo testing of a candidate anti-inflammatory substance, comprising the steps of:
a) providing a sample, preferably a serum or a plasma sample, from a patient in need of an anti-inflammatory treatment to whom the said candidate substance has been administered prior to collecting the said sample. b) performing the method according to claim 7 on the said patient, and
c) determining the anti-inflammatory effect of the said candidate substance on the said patient.

15. A kit for determining the inflammatory state of a subject, the said kit comprising (i) means necessary for measuring the amount value of at least one peptide according to claim 1 in a sample collected from the said subject and (ii) a standard comprising one or more peptides.

16. The kit according to claim 15, further comprising means necessary for measuring the amount value of human endocan of SEQ ID N^{o} 1 in the said subject's sample.

17. A method for the in vitro screening of anti-inflammatory candidate substances comprising the steps of:
a) providing an assay sample comprising cathepsin G and a measured amount value of human endocan of SEQ ID N^{o} 1, in the presence of the candidate substance to be tested; and
b) determining the amount value of said human endocan of SEQ ID N^{o} 1 or peptide according to claim 1, in the said assay sample after a suitable time.
